(19) United States
(12) Patent Application Publication
(10) Pub. No.: US 2026/0237463 A1
(43) Pub. Date: Aug. 13, 2026

(54) SYSTEMS AND METHODS FOR SEED TRAIN OPTIMIZATION

(71) Applicant: Amgen Inc., Thousand Oaks, CA (US)

(72) Inventors: Yi Li, Van Nuys, CA (US); Chun Chen, Newbury Park, CA (US)

(73) Assignee: Amgen Inc., Thousand Oaks, CA (US)

(21) Appl. No.: 19/153,560

(22) PCT Filed: Mar. 12, 2024
(86) PCT No.: PCT/US2024/019521

§ 371 Date: Aug. 4, 2025

Related U.S. Application Data
(60) Provisional application No. 63/489,958, filed on Mar. 13, 2023.

(51) Int. Cl. G16B 40/00

(52) U.S. Cl. CPC G16B 40/00

(57) ABSTRACT

Techniques for determining parameters for a seed train process for growing a culture of cells. The techniques include obtaining a specification of seed train process constraints for multiple stages of the seed train process; determining the seed train process parameters using the seed train process constraints, the seed train process parameters comprising a respective set of parameters for each particular stage of multiple stages of the seed train process; determining, using historical cell growth data and the seed train process parameters, a likelihood indicative of whether performing the seed train process using the seed train process parameters will satisfy seed train process success criteria; and outputting the set of seed train process parameters for each stage of the multiple stages of the seed train process and the likelihood indicative of whether performing the seed train process will satisfy the seed train process success criteria.

### RELATED APPLICATIONS

This application claims the benefit of priority under 35 U.S.C. § 119(e) to U.S. Application Ser. No. 63/489,958, filed Mar. 13, 2023, entitled “SYSTEMS AND METHODS FOR SEED TRAIN OPTIMIZATION”, the entire contents of which is incorporated by reference herein.

### BACKGROUND

Biological products are used to treat, prevent, and diagnose various medical conditions and diseases. Biological products may be produced by cells specifically engineered for their production. Some biological products include polypeptides (e.g., therapeutic proteins, monoclonal antibodies, vaccines, etc.), nucleic acids (e.g., messenger RNA (mRNA), small interfering RNA (siRNA), microRNA, etc.), or viral vectors (e.g., lentiviral vectors, adenoviral vectors, recombinant adeno-associated virus (rAAv) vectors, etc.).

Growing a cell culture that is used to produce biological products helps to increase their production. Such a culture is typically grown using a “seed train process,” which refers to the process used to progressively scale a culture from a small volume of cells to a larger volume of cells. A seed train process typically includes multiple stages, each of which corresponds to a cultivation system used to expand the culture to a volume larger than that achieved during the preceding stage.

### SUMMARY

Some embodiments provide for a method for determining seed train process parameters for a seed train process for growing a culture of cells, the seed train process having multiple stages, the method performed using at least one software application program comprising a common interface module, a parameter optimization module, and a simulation module. In some embodiments, the method includes using at least one computer hardware processor to perform: obtaining, using the common interface module, a specification of seed train process constraints for the multiple stages of the seed train process; determining the seed train process parameters using the parameter optimization module and the seed train process constraints, the seed train process parameters comprising a respective set of parameters for each particular stage of the multiple stages of the seed train process, the determining comprising: determining, for each particular stage of the multiple stages and using the parameter optimization module and the seed train process constraints, the respective set of parameters for growing the culture during the particular stage; determining, using the simulation module, historical cell growth data, and the determined seed train process parameters, a likelihood indicative of whether performing the seed train process using the determined seed train process parameters will satisfy seed train process success criteria; and outputting the respective set of seed train process parameters determined for each stage of the multiple stages of the seed train process and the likelihood indicative of whether performing the seed train process using the determined seed train process parameters will satisfy the seed train process success criteria.

Some embodiments provide for a system comprising: at least one computer hardware processor; and at least one non-transitory computer-readable storage medium storing processor-executable instructions that, when executed by the at least one computer hardware processor, cause the at least one computer hardware processor to perform a method for determining seed train process parameters for a seed train process for growing a culture of cells, the seed train process having multiple stages, the method performed using at least one software application program comprising a common interface module, a parameter optimization module, and a simulation module. In some embodiments, the method includes using at least one computer hardware processor to perform: obtaining, using the common interface module, a specification of seed train process constraints for the multiple stages of the seed train process; determining the seed train process parameters using the parameter optimization module and the seed train process constraints, the seed train process parameters comprising a respective set of parameters for each particular stage of the multiple stages of the seed train process, the determining comprising: determining, for each particular stage of the multiple stages and using the parameter optimization module and the seed train process constraints, the respective set of parameters for growing the culture during the particular stage; determining, using the simulation module, historical cell growth data, and the determined seed train process parameters, a likelihood indicative of whether performing the seed train process using the determined seed train process parameters will satisfy seed train process success criteria; and outputting the respective set of seed train process parameters determined for each stage of the multiple stages of the seed train process and the likelihood indicative of whether performing the seed train process using the determined seed train process parameters will satisfy the seed train process success criteria.

Some embodiments provide for at least one non-transitory computer-readable storage medium storing processor-executable instructions that, when executed by at least one computer hardware processor, cause the at least one computer hardware processor to perform a method for determining seed train process parameters for a seed train process for growing a culture of cells, the method performed using at least one software application program comprising a common interface module, a parameter optimization module, and a simulation module. In some embodiments, the method includes using at least one computer hardware processor to perform: obtaining, using the common interface module, a specification of seed train process constraints for the multiple stages of the seed train process; determining the seed train process parameters using the parameter optimization module and the seed train process constraints, the seed train process parameters comprising a respective set of parameters for each particular stage of the multiple stages of the seed train process, the determining comprising: determining, for each particular stage of the multiple stages and using the parameter optimization module and the seed train process constraints, the respective set of parameters for growing the culture during the particular stage; determining, using the simulation module, historical cell growth data, and the determined seed train process parameters, a likelihood indicative of whether performing the seed train process using the determined seed train process parameters will satisfy seed train process success criteria; and outputting the respective set of seed train process parameters determined for each stage of the multiple stages of the seed train process and the likelihood indicative of whether performing the seed train process using the determined seed train process parameters will satisfy the seed train process success criteria.

In some embodiments, determining, for each particular stage of the multiple stages, the respective set of parameters for growing the culture during the particular stage comprises: determining at least one seed train process parameter selected from the group consisting of: a viable cell density, an indication of whether the particular stage is included in the seed train process, a targeted duration of the seed train process, a batch medium volume, a working volume per vessel, and a number of vessels.

In some embodiments, obtaining the specification of the seed train process constraints comprises: obtaining a specification of at least one seed train process constraint selected from the group consisting of: a target viable cell density range, a target working volume range, a split ratio culture growth process constraint, and a batch media volume range.

In some embodiments, the historical cell growth data comprises data indicative of a doubling time associated with the growth of one or more cultures of cells.

In some embodiments, determining the seed train process parameters using the parameter optimization module comprises determining the seed train process parameters using a genetic algorithm.

In some embodiments, determining the likelihood indicative of whether performing the seed train process using the determined seed train process parameters will satisfy the seed train process success criteria comprises performing Monte Carlo simulations using the historical cell growth data.

In some embodiments, the seed train process success criteria comprise, for each stage of the multiple stages of the seed train process: a first criterion that an expected duration needed to grow the culture during the particular stage does not exceed a threshold duration for growing the culture during the particular stage; and a second criterion that an expected viable cell density resulting from growing the culture during the particular stage does not exceed a threshold viable cell density resulting from growing the culture during the particular stage.

In some embodiments, determining the respective set of parameters for each particular stage of the multiple stages comprises: determining, using the respective set of parameters, the expected duration needed to grow the culture of cells during the particular stage; and determining whether the expected duration exceeds the respective threshold duration for the particular stage.

In some embodiments, determining the respective set of parameters for each particular stage of the multiple stages comprises: determining, using the respective set of parameters, the expected viable cell density resulting from growing the culture of cells during the particular stage; and determining whether the expected viable cell density exceeds the respective threshold viable cell density for the particular stage.

In some embodiments, outputting the set of seed train process parameters for each stage of the multiple stages of the seed train process and the likelihood indicative of whether performing the seed train process will satisfy the seed train process success criteria comprises: generating a graphical user interface (GUI) using the common interface module; and displaying the set of seed train process parameters and/or the likelihood through the generated GUI.

In some embodiments, displaying the set of seed train process parameters through the common interface module comprises: displaying a visual indication of whether a seed train process parameter of the set of seed train process parameters violates a seed train process constraint of the seed train process constraints.

In some embodiments, determining the seed train process parameters comprises: determining the seed train process parameters based on a set of estimate culture doubling times included in the seed train process constraints, the set of estimate culture doubling times including an estimate culture doubling time for each of the multiple stages of the seed train process; and determining whether performing the seed train process using the determined seed train process parameters will satisfy the seed train process success criteria. Some embodiments further comprise displaying, through a graphical user interface (GUI) generated by the common interface module, a visual indication of a result of determining whether performing the seed train process using the seed train process parameters will satisfy the seed train process success criteria.

Some embodiments further comprise: comparing the determined likelihood to a likelihood threshold, wherein outputting the set of seed train process parameters for each stage of the multiple stages of the seed train process comprises outputting determined the seed train process parameters upon determining that the likelihood exceeds the likelihood threshold.

In some embodiments, outputting the set of seed train process parameters for each stage of the multiple stages of the seed train process comprises: when the likelihood indicative of whether performing the seed train process using the determined seed train process parameters will satisfy the seed train process success criteria satisfies a likelihood threshold, outputting a recommendation to perform the seed train process using the set of seed train process parameters for each stage of the multiple stages of the seed train process.

In some embodiments, the at least one software application program further comprises a seed train process automation module, and outputting the set of seed train process parameters for each stage of the multiple stages of the seed train process and the likelihood indicative of whether performing the seed train process will satisfy the seed train process success criteria comprises: transmitting the set of seed train process parameters and/or the likelihood to the seed train process automation module; and using the seed train process automation module to cause a seed train process automation system to perform a particular stage of the multiple stages of the seed train process according to the respective set of seed train process parameters.

Some embodiments further comprise: obtaining, using the common interface module, input indicative of a result of growing the culture of cells during a first stage of the multiple stages of the seed train process; determining, using the parameter optimization module, the input, and the seed train process constraints, updated seed train process parameters for subsequent stages of the seed train process; and outputting the updated seed train process parameters.

Some embodiments further comprise: obtaining, using the common interface module, a specification of second seed train process constraints for multiple stages of a second seed train process; determining, using the parameter optimization module and the second seed train process constraints, second seed train process parameters for the second seed train process; determining, using the simulation module, the historical cell growth data, and the second seed train process parameters, a second likelihood indicative of whether performing the second seed train process, using the second seed train process parameters, will satisfy second seed train process criteria; and outputting the second seed train process parameters and the second likelihood indicative of whether performing the second seed train process, using the second seed train process parameters, will satisfy the second seed train process criteria.

In some embodiments, determining the seed train process parameters using the parameter optimization module comprises: determining, using an objective function, a first score for first candidate seed train process parameters; determining, using the objective function, a second score for second candidate seed train process parameters; comparing the first score and the second score; and selecting, based on the result of the comparing, the seed train process parameters from among the first candidate seed train process parameters and the second candidate seed train process parameters.

In some embodiments, the seed train process parameters comprise a first set of seed train process parameters for a first stage of the multiple stages of the seed train process, and determining the likelihood indicative of whether performing the seed train process using the seed train process parameters will satisfy the seed train process success criteria comprises: determining, for the first stage of the seed train process, a first likelihood indicative of whether growing the culture of cells during the first stage, using the first set of seed train parameters for the first stage, will satisfy first criteria of the seed train process success criteria.

In some embodiments, determining the likelihood indicative of whether performing the seed train process using the seed train process parameters will satisfy the seed train process success criteria comprises: simulating a first set of cell growth values using the historical cell growth data, the first set of cell growth values including a first historical cell growth value for each of the multiple stages of the seed train process; predicting, using the seed train process parameters and the first set of cell growth values, a first outcome of performing the seed train process, wherein the first outcome is indicative of whether performing the seed train process using the determined seed train process parameters will satisfy the seed train process success criteria; simulating a second set of cell growth values using the historical cell growth data, the second set of cell growth values including a second historical cell growth value for each of the multiple stages of the seed train process; predicting, using the seed train process parameters and the second set of cell growth values, a second outcome of performing the seed train process, wherein the second outcome is indicative of whether performing the seed train process using the seed train process parameters will satisfy the seed train process success criteria; and determining the likelihood based on the predicted first and second outcomes.

In some embodiments, determining the likelihood based on the predicted first and second outcomes comprises: determining a number of predicted outcomes that indicate that performing the seed train process using the seed train process parameters and respective historical cell growth data will satisfy the seed train process success criteria; and determining a ratio of the number of predicted outcomes to a total number of predicted outcomes.

In some embodiments, the seed train process success criteria comprise, for each stage of the multiple stages of the seed train process: a first criterion that an expected duration needed to grow the culture during the particular stage does not exceed a threshold duration for growing the culture during the particular stage, predicting the first outcome comprises, for each particular stage of the multiple stages: determining, using the respective set of seed train process parameters for the particular stage and a historical cell growth value of the first set of historical cell growth values, a first expected duration needed to grow the culture of cells during the particular stage of the seed train process; and comparing the first expected duration to the threshold duration, and predicting the second outcome comprises, for each particular stage of the multiple stages: determining, using the respective set of seed train process parameters for the particular stage and a historical cell growth value of the second set of historical cell growth values, a second expected duration needed to grow the culture of cells during the particular stage of the seed train process; and comparing the second expected duration to the threshold duration.

In some embodiments, the seed train process success criteria comprise, for each stage of the multiple stages of the seed train process: a second criterion that an expected viable cell density resulting from growing the culture during the particular stage does not exceed a threshold viable cell density resulting from growing the culture during the particular stage, predicting the second outcome comprises, for each particular stage of the multiple stages: determining, using the respective set of seed train process parameters for the particular stage and a historical cell growth value of the first set of historical cell growth values, a first expected viable cell density resulting from growing the culture of cells during the particular stage of the seed train process; and comparing the first expected viable cell density to the threshold viable cell density, and predicting the second outcome comprises, for each particular stage of the multiple stages: determining, using the respective set of seed train process parameters for the particular stage and a historical cell growth value of the second set of historical cell growth values, a second expected viable cell density resulting from growing the culture of cells during the particular stage of the seed train process; and comparing the second expected viable cell density to the threshold viable cell density.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A is a diagram depicting an illustrative technique 100 for determining seed train process parameters for a seed train process, according to some embodiments of the technology described herein.

FIG. 1B is a block diagram of an example system 160 for determining seed train process parameters for a seed train process, according to some embodiments of the technology described herein.

FIG. 2 is a flowchart of an illustrative process 200 for determining seed train process parameters for a seed train process, according to some embodiments of the technology described herein.

FIG. 3A is a flowchart of an illustrative process 300 for determining the seed train process parameters using a parameter optimization module and seed train process constraints, according to some embodiments of the technology described herein.

FIG. 3B shows an example objective function used for determining seed train process parameters, according to some embodiments of the technology described herein.

FIG. 4 is a flowchart of an illustrative process 400 for determining a likelihood indicative of whether performing the seed train process using the seed train process parameters will satisfy seed train process success criteria, according to some embodiments of the technology described herein.

FIG. 5A-1, FIG. 5A-2, FIG. 5B-1, FIG. 5B-2, FIG. 5C-1, and FIG. 5C-2 show an example interface depicting seed train process parameters and seed train process constraints, according to some embodiments of the technology described herein.

FIG. 5D shows an example interface depicting predictions of whether performing the seed train process using the seed train process parameters will satisfy the seed train process success criteria, according to some embodiments of the technology described herein.

FIG. 6 shows example historical cell growth data for doubling times.

FIG. 7A and FIG. 7B show example doubling time data, from which historical cell growth data is derived, according to some embodiments of the technology described herein.

FIG. 8A and FIG. 8B show a higher overall success rate of performing a seed train process, according to embodiments of the technology described herein, as compared to performing a manually-designed seed train process.

FIG. 8C shows that performing a 500 L seed train process, according to embodiments of the technology described herein, results in a success rate of over 80% for each stage of the seed train process.

FIG. 8D shows that performing a 2 kL seed train process, according to embodiments of the technology described herein, results in a success rate of over 70% for each stage of the seed train process.

FIG. 9 is a schematic diagram of an illustrative computing device with which aspects described herein may be implemented.

### DETAILED DESCRIPTION

The inventors have developed techniques for designing and implementing an optimized seed train process for growing a culture of cells. In some embodiments, the techniques include determining seed train process parameters for the seed train process. For example, a set of seed train process parameters may be determined for each of multiple stages of the seed train process. In some embodiments, the seed train process parameters are determined using a parameter optimization module and seed train process constraints for each of the multiple stages of the seed train process. After determining the seed train process parameters, the techniques include determining, using a simulation module and historical cell growth data, a likelihood indicative of whether performing the seed train process using the determined seed train process parameters will satisfy various seed train process success criteria such as, for example, a success criterion that the estimated duration of implementing a stage of the seed train process does not exceed threshold duration and a success criterion that an estimated viable cell density resulting from growing the culture during a stage of the seed train process does not exceed a threshold viable cell density.

While a small vial of cells could be used to produce a biological product, this would be highly inefficient for producing industrial-scale quantities of such a product. Accordingly, the culture of cells is grown to a volume that can support industrial-scale production. For example, the culture of cells may be grown to a volume that can be used to inoculate a bioreactor, which is a large system that supports a biologically active environment for large-scale bioproduction. Growing the cell culture to such a volume involves progressively scaling the culture from the initial volume to the target volume. Depending on the target volume, this process can take up to days or weeks.

A “seed train process” refers to the process used to progressively scale a culture from a small volume of cells to a larger volume of cells. A seed train process typically includes multiple stages (“multi-stage seed train process”), each of which corresponds to a cultivation system used to grow the culture during the particular stage. For example, the first stage of a seed train process may utilize a 250 mL shake flask. During the first stage, the cells are grown in the 250 mL shake flask until the culture satisfies particular criteria, such as achieving a target viable cell density. The culture is then transferred to a different container, such as a 1000 mL shake flask, for growth during the second stage. As the seed train process progresses, the culture of cells continues to expand until the target volume is attained.

Various factors affect the quality of a seed train process. Examples of such factors include the vessels selected, the volume of culture medium used to fill the selected vessels, the ratio of fresh medium to passaged cell culture, the duration of the culture, and the apparent growth rate. In designing a seed train process, many of these factors can be adjusted to control the growth of the culture. For example, the designer of the seed train process (e.g., a user or an automated system) may select the vessel for each stage of the seed train process and the volume of culture medium for filling each vessel. However, other factors cannot be controlled. For example, cell growth rate varies between different stages and different cultures. Furthermore, the cell growth rate is complex in that it depends on several external factors such as, process scales, vessel selection, seeding densities, substrate, and metabolite concentrations.

Because cell growth rate directly impacts the time needed to complete a stage of the seed train process, and due to the variations among different stages and cultures, it is challenging to predict how much time will be needed to complete the seed train process or a particular stage of a seed train process. For example, cells growing at an unexpectedly low cell growth rate will require more time to achieve the target volume, leading to delays. Such delays can cause scheduling issues for the manufacturing facility where the seed train process is being performed. By contrast, cells growing at an unexpectedly high cell growth rate will require less time to achieve the target volume but may lead to cell overgrowth if the culture is not being monitored. When a culture becomes overgrown it leads to cell death and, in many cases, the culture must be discarded. This not only wastes resources, but also results in significant delays incurred by regrowing the culture.

Conventional techniques for designing seed train processes are empirical. They involve designing later stages of the seed train process based on observations made during earlier stages of the seed train process. For example, such techniques involve growing the culture during a particular stage of the seed train process, recording observations about culture growth (e.g., cell growth rate) during that stage, and using those observations to design the next stage of the seed train process. While such techniques may facilitate the successful expansion of a high-quality cell culture by preventing cell overgrowth, they do not allow for an estimation of the duration of the seed train process. First, such techniques are still subject to the delays introduced by unexpected changes in cell growth rate across different stages of the seed train process. Second, many parameters that affect the duration of the seed train process, such as number of stages, vessel selection, and split ratio, for example, are selected during the middle of the seed train process, making it challenging to predict, ahead of time, how long different stages of the seed train process will take to complete.

Accordingly, the inventors have developed techniques that address the above-described limitations of conventional seed train process design techniques. In some embodiments, the techniques include: (a) obtaining a seed train process constraints for multiple stages of a seed train process, (b) determining seed train process parameters using the seed train process constraints and an optimization module, (c) determining, using a simulation module and historical cell growth data, the likelihood indicative of whether performing the seed train process using the seed train process parameters will satisfy seed train process success criteria, and (d) outputting the determined seed train process parameters and the determined likelihood.

The term “seed train process constraints” refers to aspects of the seed train process that cannot be varied. In some embodiments, they are defined by the facility where the seed train process is performed. For example, the duration of the seed train process may be limited by the schedule of the facility where the seed train process is performed. Additionally, or alternatively, in some embodiments, the seed train process constraints are defined by the equipment available for performing the seed train process. For example, the type, number, and/or volume of available containers may restrict, at each stage, the volume of media that can be supplied to the cell culture. Additionally, or alternatively, in some embodiments, the seed train process constraints are defined by the cell culture itself. For example, different cell lines grow at different rates. The growth rate may be a constraint of the seed train process since it cannot be changed. In some embodiments, the growth rate and/or the doubling time of a particular cell line may be estimated based on historical cell growth data. Example seed train process constraints are listed in Table 1.

The term, “seed train process parameters” may refer to aspects of the seed train process that can be varied. The seed train process parameters may be selected within the seed train process constraints. For example, the seed train process constraints may define the upper and lower bounds of a particular aspect of the seed train process (e.g., medium volume), and the seed train process parameter may be selected from within those bounds. Example seed train process parameters are listed in Table 2.

The term “success criteria” may refer to one or more criteria defining one or more successful outcomes of implementing the seed train process. In some embodiments, the success criteria include a criterion that an expected duration needed to grow the culture during a particular stage of the seed train process does not exceed a threshold duration for growing the culture during the particular stage. If the duration criterion is satisfied, in some embodiments, this may indicate that no more than a threshold amount of extra time is needed to grow the culture of cells during the particular stage. If the duration criterion is not satisfied, this may indicate that performing the seed train process according to the seed train process constraints and the selected parameters may potentially disrupt the schedule of the facility and/or the timing of downstream processes. Additionally, or alternatively, in some embodiments, the success criteria include a criterion that an expected viable cell density resulting from growing the culture during a particular stage of the seed train process does not exceed a threshold viable cell density. In some embodiments, if the expected viable cell density does not exceed the threshold (e.g., the criterion is satisfied), this may indicate that growing the culture during the particular stage according to the seed train process constraints and the selected parameters will not result in cell culture overgrowth. By contrast, if the expected viable cell density exceeds the threshold viable cell density, this may indicate that growing the culture during the particular stage, according to the seed train process constraints and selected parameters, may result in cell culture overgrowth.

The techniques developed by the inventors improve upon conventional seed train process design techniques because they involve determining seed train process parameters that, when used to implement one or more stages of the seed train process, are estimated to result in the successful completion of the seed train process under average cell growth conditions. Such techniques allow for the design of the seed train process before starting the seed train process, thereby enabling manufacturing facilities to estimate accurately the time need to complete the seed train process and to develop schedules accordingly. This is much more efficient than conventional techniques which design the seed train process during implementation of the seed train process, thereby preventing accurate estimates of how much time will be needed complete the seed train process.

Furthermore, the techniques developed by the inventors improve upon conventional techniques because they account for variability in historical cell growth rates to predict the likelihood of successfully completing the seed train process using the determined seed train process parameters. Therefore, prior to implementing the seed train process, it is possible to estimate whether the seed train process will be successful if the cell growth rates are slower or faster than average. Such information may be used to determine whether to proceed with implementing the seed train process using the determined parameters, determine updated parameters, or cancel the seed train process altogether. Accordingly, such techniques help to ensure compliance with facility scheduling and reduce the waste in resources and time.

It should be appreciated that the techniques described herein may be implemented in any of numerous ways, as the techniques are not limited to any particular manner of implementation. Examples of details of implementation are provided herein solely for illustrative purposes. Furthermore, the techniques disclosed herein may be used individually or in any suitable combination, as aspects of the technology described herein are not limited to the use of any particular technique or combination of techniques.

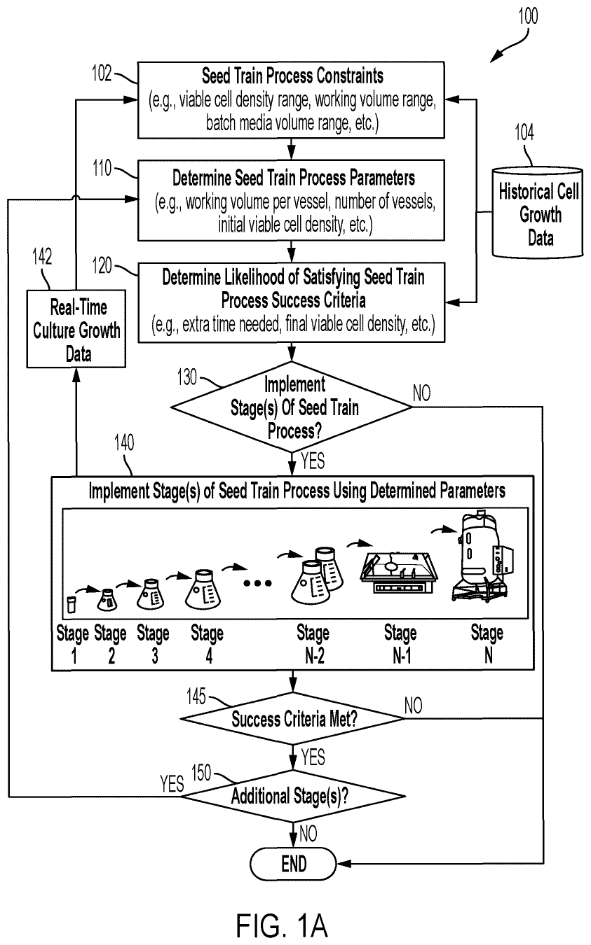

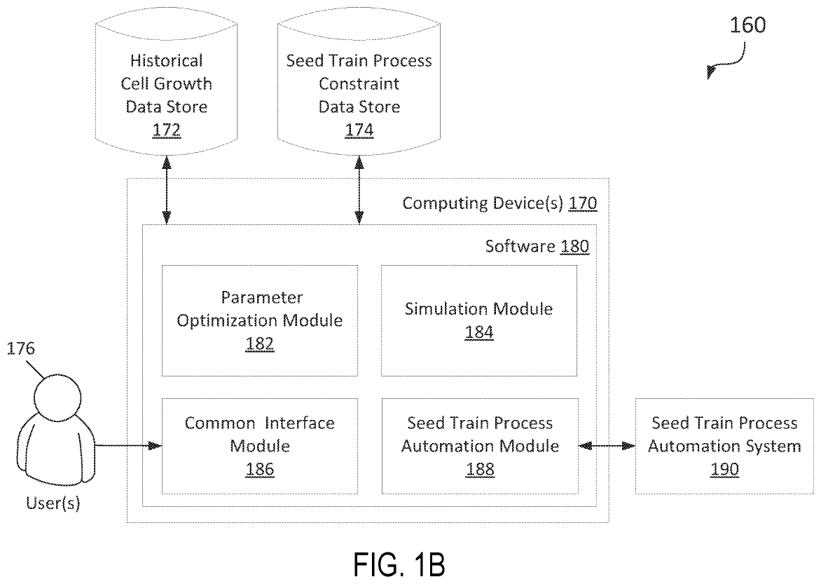

FIG. 1A is a diagram depicting an illustrative technique 100 for designing a seed train process, according to some embodiments of the technology described herein. In some embodiments, the technique 100 includes (a) determining, at act 110, seed train process parameters based on seed train process constraints 102, (b) determining, at act 120, based on historical cell growth data 104, the likelihood of satisfying seed train process success criteria as a result of performing one or more stages of the seed train process according to the determined parameters, and (c) determining, at act 130, based on the likelihood of satisfying the success criteria, whether to implement one or more stages of the seed train process. In some embodiments, technique 100 ends if it is determined at act 130 that the one or more stages of the seed train process should not be implemented. In some embodiments, upon determining that the one or more stages should be implemented, technique 100 includes implementing the one or more stages of the seed train process at act 140. Based on the outcome of implementing the one or more stages of the seed train process, act 145 includes determining whether the outcome satisfies the success criteria. If the outcome satisfies the success criteria, then act 150 includes determining whether there are additional stages of the seed train process. If there are additional stages of the seed train process, then one or more acts of technique 100 may be repeated for the next stage. Otherwise, technique 100 ends.

As described herein, a seed train process is used to scale a culture from a small volume of cells to a larger volume of cells. For example, at the beginning of the seed train process, the culture of cells may occupy a container having a volume of 25 mL, 50 mL, 100 mL, 150 mL, 200 mL, 250 mL, 300 mL, 400 mL, 500 mL, between 10 mL and 1000 mL, between 25 mL and 500 mL, between 200 mL and 400 mL, or any other suitable volume, as aspects of the technology described herein are not limited in this respect. During the seed train process, the culture of cells may be expanded to achieve any suitable volume. For example, by the end of the seed train process, the culture of cells may occupy a container that is factor of 2, 5, 10, 25, 50, 75, 100, 150, 200, 300, 500, 1,000, 2,000, 5,000, 10,000, between 1.5 and 50,000, between 2 and 10,000, between 25 and 5,000, or any other suitable factor times the size of the initial container, as aspects of the technology described herein are not limited in this respect.

In some embodiments, the seed train process includes multiple stages. For example, as shown at act 140 of technique 100, the seed train process includes stage 1 through stage N−1, where N is any suitable number, as aspects of the technology described herein are not limited to any particular number of seed train process stages. As described herein, in some embodiments, the number of stages is a seed train process parameter determined at act 110 of technique 100.

In some embodiments, performing the seed train process involves growing the culture of cells according determined seed train process parameters. In some embodiments, each stage of the seed train process is associated with a respective set of seed train process constraints and/or seed train parameters. For example, the first stage of the seed train process may be associated with a first set of constraints, and, during the first stage, the culture of cells may be grown using a first set of parameters. The Nth stage of the seed train process may be associated with an Nth set of constraints, and, during the Nth stage, the culture of cells may be grown using an Nth set of parameters. The first set of constraints and the Nth set of constraints may be the same (e.g., include all of the same constraints) or different (e.g., include some or none of the same constraints). The first set of parameters and the Nth set of parameters may be the same (e.g., include all of the same parameters) or different (e.g., includes some or none of the same parameters).

In some embodiments, the seed train process constraints 102 are aspects of the seed train process that cannot be varied. In some embodiments, they are defined by the facility where the seed train process is performed. For example, the duration of the seed train process may be limited by the schedule of the facility where the seed train process is performed. Additionally, or alternatively, in some embodiments, the seed train process constraints 102 are defined by the equipment available for performing the seed train process. For example, the type, number, and/or volume of available containers may restrict, at each stage, the volume of media that can be supplied to the cell culture. Additionally, or alternatively, in some embodiments, the seed train process constraints 102 are defined by the cell culture itself. For example, different cell lines grow at different rates. The growth rate may be a constraint of the seed train process since it cannot be changed. In some embodiments, the growth rate and/or the doubling time of a particular cell line may be estimated based on historical cell growth data obtained from historical cell growth data store 172.

Nonlimiting examples of seed train process constraints are listed in Table 1. It should be appreciated that the seed train process may be associated with additional or alternative seed train process constraints, as aspects of the technology described herein are not limited in this respect. In some embodiments, an example seed train process constraint listed in Table 1 may apply to one, some, or all of the stages of the seed train process. In some embodiments, after one or more stages of the seed train process are performed, the seed train process constraints 102 may be updated to include the real-time culture growth data 142, such as the doubling time and growth constant of the cells during the completed stages of the seed train process. Additional, or alternative, examples of seed train process constraints are described herein including at least with respect to FIG. 5A-1-FIG. 5C-2.

TABLE 1
Example seed train process constraints.

| Seed Train Process Constraint | Description |
|---|---|
| Standard Batch Medium Volume | The recommended volume of the cell culture medium |
| | that is added to the vessel before inoculation. |
| Batch Volume Lower Limit | The lower limit of the batch medium volume. |
| Batch Volume Upper Limit | The upper limit of the batch medium volume. |
| Standard Working Volume | The recommended total cell culture volume in the vessel. |
| Working Volume Lower Limit | The lower limit of the working volume. |
| Working Volume Upper Limit | The upper limit of the working volume. |
| Standard Initial Viable Cell | The recommended VCD of the cell culture right after |
| Density (VCD) | the inoculation. |
| Initial VCD Lower Limit | The lower limit of the initial VCD. |
| Initial VCD Upper Limit | The upper limit of the initial VCD. |
| Evaporative Loss Fraction | The theoretical volume loss due to evaporation. For |
| | example, 10% evaporative volume loss in shake |
| | flasks; 5% evaporative volume loss in wave bags and |
| | bioreactors. |
| Growth Constant - Normal | The cell growth rate assuming normal doubling time. |
| | $Equal to : \frac{ln ( 2 )}{Normal Doubling Time} .$ |
| Growth Constant - Worst | The cell growth rate assuming the longest doubling |
| | $time . Equal to : \frac{ln ( 2 )}{Worst Doubling Time} .$ |
| Growth Constant - Best | The cell growth rate assuming the shortest doubling |
| | $time . Equal to : \frac{ln ( 2 )}{Best Doubling Time} .$ |
| Standard Culture Time | The duration of the cell culture. |
| Doubling Time - Normal | The doubling time under a normal growth condition |
| | for the cell line used. |
| Doubling Time - Worst | The doubling time if the cell line is under the worst |
| | growth condition. |
| Doubling Time - Best | The doubling time if the cell line is under the best |
| | growth condition. |
| Passage Date | The date on which the inoculation occurs for the stage. |
| Binary Value That Indicates | Whether the stage is included in the seed train. Certain |
| Whether A Stage Should Be | stages may be skipped for a shorter seed train. |
| Included Or Skipped | |
| Batch Medium Volume | The batch medium volume. |
| Working Volume Per Vessel | The working volume per vessel. |
| Number of Vessels | The number of vessels used for growing the culture |
| | during the stage of the seed train process. |
| Initial VCD | The VCD of the cell culture right after the inoculation. |

In some embodiments, the seed train process parameters are aspects of the seed train process that can be varied. Nonlimiting examples of seed train process parameters are listed in Table 2. However, it should be appreciated that the seed train process may be associated with additional, or alternative, seed train process parameters, as aspects of the technology described herein are not limited in this respect. In some embodiments, an example seed train process parameter listed in Table 2 may apply to one, some, or all of the stages of the seed train process. In some embodiments, one or more seed train process parameters listed in Table 2 may be calculated based on one or more other seed train process parameters and/or seed train process constraints. For example, Table 3 lists equations for calculating at least some of the seed train process parameters in Table 2. Additional, or alternative, examples of seed train process parameters are described herein including at least with respect to FIG. 5B-1-FIG. 5C-2.

TABLE 2
Example seed train process parameters.

| Seed Train Process Parameter | Description |
|---|---|
| Cell Bank Vial VCD | The VCD of the cell bank that is used for inoculating |
| | the vessel(s) at the first passage of the seed train |
| | process. |
| Binary Value That Indicates | Whether the stage is included in the seed train. Certain |
| Whether A Stage Should Be | stages may be skipped for a shorter seed train. |
| Included Or Skipped | |
| Batch Medium Volume | The batch medium volume. |
| Working Volume Per Vessel | The working volume per vessel. |
| Number of Vessels | The number of vessels used for growing the culture |
| | during a stage of the seed train process. |
| Initial VCD | The VCD of the cell culture right after the inoculation. |
| Total Volume After Loss | The cell culture volume after accounting for the |
| | evaporative loss and the sampling loss. For example, |
| | sampling loss may be assumed to be 5 mL in shake |
| | flasks, 30 mL in wave bags, and 500 mL in bioreactors. |
| | For a 60 mL shake flask, 10 mL total loss, including |
| | evaporation and sampling, may be assumed. |
| Minimum Final VCD Required | The minimum VCD at the end of the stage that is |
| | needed for inoculating the next stage. Determined by |
| | taking the larger of: (a) the minimum cell mass to |
| | achieve the target VCD and working volume of the next |
| | passage; and (b) the minimum cell mass to achieve a |
| | split ratio of at least 2 (e.g., volume of fresh media is at |
| | least 2 times the volume of the cell culture). |
| Predicted Final VCD - Normal | The predicted VCD at the end of the stage using the |
| | normal growth rate. |
| Split Ratio | The ratio of fresh medium volume to the cell culture |
| | volume used for passage. |
| Time Needed - Normal | The time needed for the cell culture to reach the |
| | minimum VCD for passage. Calculated assuming |
| | exponential cell growth using the defined normal |
| | growth rate. |

In some embodiments, varying the seed train process parameters affects the outcome of performing the seed train process. For example, varying the seed train process parameters may affect the time needed to complete a stage of the seed train process. Additionally, or alternatively, varying the seed train process parameters may affect the viable cell density resulting from growing the culture according to the seed train process parameters.

In some embodiments, designing a seed train process includes estimating the outcome of performing a seed train process according to seed train process parameters and seed train process constraints. This may include, for example, estimating the duration needed to grow the culture during a particular stage of the seed train process. In some embodiments, the duration of a stage of a seed train process may depend on one or multiple seed train process parameters and/or constraints. For example, the doubling time of a cell line, which may be estimated based on historical cell growth data (e.g., historical cell growth data 104), may affect the duration of a particular stage of the seed train process. Given the variation in doubling time, it may be beneficial to (a) estimate a duration based on the normal doubling time for the cell line (e.g., the average doubling time determined from the historical cell growth data 104) and (b) estimate a duration based on the worst longest doubling time for the cell line based on the historical cell growth data 104. In some embodiments, the estimated durations may provide an indication as to whether the seed train process will comply with the schedule of the facility and/or whether extra time will be needed to perform the seed train process.

Additionally, or alternatively, estimating the outcome of performing a seed train process according to seed train process parameters and seed train process constraints may include estimating the final viable cell density resulting from growing the culture during a particular stage. In some embodiments, the estimated final viable cell density provides an indication as to whether growing the culture of cells during the particular stage will result in overgrowth of the cell culture, which could lead to cell death. In some embodiments, the final viable cell density also depends on the doubling time of the cell line. Accordingly, in some embodiments, the final viable cell density may be estimated based on the normal doubling time for the cell line (e.g., the average doubling time determined from the historical cell growth data 104) and/or based on the shortest doubling time.

In some embodiments, the estimated outcomes of the seed train process are evaluated to determine whether they satisfy one or more “success criteria.” In some embodiments, the success criteria include a criterion that an expected duration needed to grow the culture during a particular stage does not exceed a threshold duration for growing the culture during the particular stage. In some embodiments, the threshold duration includes any suitable duration, as aspects of the technology are not limited in this respect. For example, the threshold duration may be defined by a seed train process constraint (e.g., standard culture time). Additionally, or alternatively, in some embodiments, the success criteria include a criterion that the extra time needed (e.g., relative to an allotted time) to grow the culture during a particular stage does not exceed a threshold value. For example, the threshold value may be 0, 2, 4, 8, 12, 20, 40, or any other suitable number of hours, as aspects of the technology are not limited in this respect. If the duration criterion is not satisfied, this may indicate that performing the seed train process according to the seed train process constraints and the selected parameters may potentially disrupt the schedule of the facility and/or the timing of downstream processes.

Additionally, or alternatively, in some embodiments, the success criteria include a criterion that an expected viable cell density resulting from growing the culture during a particular stage of the seed train process does not exceed a threshold viable cell density. In some embodiments, if the expected viable cell density does not exceed the threshold (e.g., the criterion is satisfied), this may indicate that growing the culture during the particular stage according to the seed train process constraints and the selected parameters will not result in cell culture overgrowth. By contrast, if the expected viable cell density exceeds the threshold viable cell density, this may indicate that growing the culture during the particular stage, according to the seed train process constraints and selected parameters, may result in cell culture overgrowth. In some embodiments, the threshold viable cell density may depend on the container being used to grow the culture during the particular stage.

In some embodiments, technique 100 includes determining, at act 110, seed train process parameters for implementing one or more stages of the seed train process. In some embodiments, the seed train process parameters are determined such that the estimated outcomes of performing the seed train process according to the determined parameters satisfy one or more success criteria. By determining seed train process parameters that are expected to result in outcomes that satisfy the success criteria, the technique 100 may be used to design a seed train process that avoids cell culture overgrowth and that complies with time constraints (e.g., of the facility whether the seed train process is performed). Additionally, or alternatively, in some embodiments, the seed train process parameters are determined such that the estimated outcomes of performing the seed train process according to the determined parameters are more desirable than estimated outcomes of performing the seed train process according to other seed train process parameters. For example, a more desirable estimated outcome may include a relatively short duration of performing one or more stages of the seed train process, and/or a relatively low viable cell density, as compared to other expected durations and/or viable cell densities.

In some embodiments, determining the seed train process parameters at act 110 includes (a) estimating, for each of multiple sets of candidate seed train process parameters, outcomes of performing the seed train process according to the candidate set of parameters, (b) comparing the estimated outcomes, and (c) determining the seed train process parameters based on a result of the comparison. Determining the seed train process parameters may include selecting the candidate parameters that result in estimated outcomes that satisfy one or more success criteria and/or selecting the candidate parameters that result in more desirable outcome(s) than the other candidate parameters. In some embodiments, the seed train process parameters are determined using a parameter optimization module, such as the parameter optimization module 182 described herein including at least with respect to FIG. 1B. In some embodiments, the parameter optimization module uses an optimization technique to determine the seed train process parameters. For example, the genetic algorithm may test candidate seed train process parameters against an objective function to determine the seed train process parameters. Techniques for determining seed train process parameters are described herein in more detail including at least with respect to FIGS. 1B, 2, and 3A-3B.

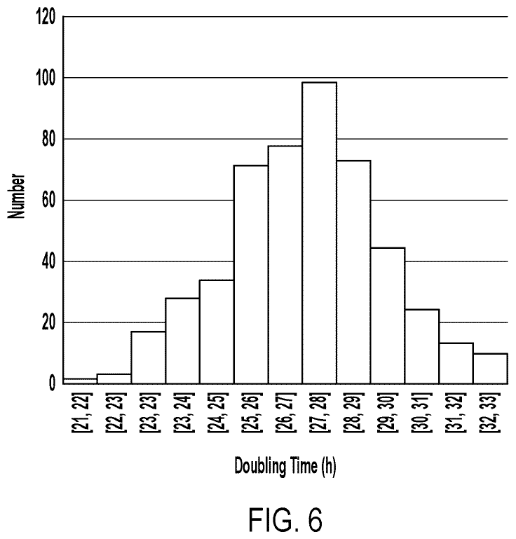

In some embodiments, as explained above, the estimated outcomes of performing a particular stage of a seed train process depend on the doubling time of the particular cell line being grown. However, doubling time varies among different cell cultures—some cultures may have relatively long doubling times, while other cultures may have relatively short doubling times. FIG. 6 shows an example distribution of doubling times for multiple cultures of the same cell line. Accordingly, while the outcome of performing the seed train process using the determined parameters may satisfy the success criteria when the doubling time of the culture is equivalent to the historical average, this may not be the case when the culture has a doubling time that is greater or less than the historical average. For example, if the true doubling time is shorter than average, then performing the seed train process according to the determined parameters may result in overgrowth. If the true doubling time is longer than average, then the duration of the seed train process may exceed an allotted duration for performing the seed train process. In other words, the outcome(s) (e.g., duration, viable cell density) of the seed train process may fail to comply with the success criteria.

Accordingly, in some embodiments, technique 100 includes determining, at act 120, the likelihood of satisfying the seed train process success criteria using the parameters determined at act 110. In some embodiments, this includes evaluating multiple input scenarios. An input scenario may include the parameters determined at act 110, and a set of doubling times randomly sampled from normal distributions calculated from the historical cell growth data 104. For example, the set of doubling times may include a doubling time for each stage of the seed train process. An input scenario may represent a potential set of conditions under which the seed train process could be performed. In some embodiments, at least 10, at least 25, at least 75, at least 100, at least 150, at least 200, at least 250, at least 300, at least 350, at least 400, at least 450, at least 500, at least 550, at least 600, at least 650, at least 700, at least 800, at least 900, at least 1,000, at least 1,500, at least 2,000, or any other suitable number of input scenarios are evaluated, as aspects of the technology are not limited in this respect.

In some embodiments, the historical cell growth data 104 includes cell doubling time data. For example, the cell doubling time data may include cell doubling time data from one or more prior seed train processes. The cell doubling time data may include cell doubling times for each of one or more stages of prior seed train processes(es). In some embodiments, the average and standard deviation of the doubling time at each stage is calculated from the data. In some embodiments, the normal distribution of doubling time at each stage is calculated by fitting average and standard deviation.

In some embodiment, determining the likelihood of satisfying the seed train process success criteria includes, for each of multiple input scenarios, (a) estimating outcome(s) (e.g., duration, final viable cell density, etc.) of performing the seed train process according to the input scenario, and (b) determining whether the estimated outcome(s) satisfy the seed train process success criteria. For example, one or more outcome(s) may be estimated for each stage of the seed train process, then compared to the seed train process success criteria for that particular stage. In some embodiments, if at least a threshold number of outcome(s) satisfy their respective success criteria, that input scenario may be considered a successful scenario. For example, if all outcome(s) satisfy their respective success criteria at each stage, the input scenario may be considered a successful scenario. In some embodiments, the likelihood of satisfying the seed train process success criteria is the ratio of the number of successful scenarios to the total number of input scenarios evaluated.

In some embodiments, a simulation module is used to determine the likelihood of satisfying the seed train process success criteria. For example, simulation module 184, described herein including at least with respect to FIG. 1B, may be used to determine the likelihood of satisfying the seed train process success criteria. In some embodiments, the simulation module performs Monte Carlo simulations to evaluate different input scenarios. The Monte Carlo simulations may be repeated any suitable number of times for a particular input scenario. For example, the Monte Carlo simulations may be repeated at least 5 times, at least 10 times, at least 25 times, at least 50 times, at least 75 times, at least 100 times, at least 125 times, at least 150 times, at least 175 times, at least 200 times, or any other suitable number of times as aspects of the technology described herein are not limited in this respect.

In some embodiments, technique 100 includes determining, at act 130, whether to implement one or more stages of the seed train process. The determination may be based on the determined likelihood of satisfying the seed train process success criteria. For example, in some embodiments, act 130 includes determining whether the likelihood of satisfying the seed train process success criteria exceeds a threshold. The threshold may be any suitable threshold, such as at least 40%, at least 50%, at least 60%, at least 70%, at least 80%, at least 85%, at least 90%, at least 95%, at least 98%, or any other suitable threshold, as aspects of the technology described herein are not limited to any particular threshold. In some embodiments, if the likelihood satisfies the threshold, then technique 100 proceeds to act 140 for implementing one or more stages of the seed train process. If the likelihood does not satisfy the threshold, then technique 100 ends or optionally, though not show, returns back to act 110 for determining seed train process parameters.

In some embodiments, act 130 is performed by a processor and/or an individual. When act 130 is performed by an individual, the likelihood of satisfying the seed train process success criteria may be output to the user. For example, likelihood may be output via the display of a user interface. Additionally, or alternatively, a processor may output a recommendation as to whether to implement one or more stages of the seed train process (e.g., via user interface). In some embodiments, the individual manually determines whether to implement the stage(s) of the seed train process based on the output likelihood of satisfying the success criteria and/or based on the recommendation of the processor. When act 130 is performed by the processor, the processor may automatically determine, based on the likelihood of satisfying the success criteria, whether to implement the one or more stage(s) of the seed train process.

In some embodiments, if it is determined that the one or more stages of the seed train process are to be implemented, technique 100 proceeds to act 140, during which the stage(s) are implemented. In some embodiments, the one or more stages include one, some, or all of the stages of the seed train process. The one or more stages may be implemented according to the seed train process parameters determined at act 110 and the seed train process constraints 102. For example, a first stage of the seed train process may be performed according to the parameters determined for the first stage and the constraints specified for the first stage. Additionally, or alternatively, multiple stages of the seed train process may be performed according to their respective seed train process parameters and constraints.

In some embodiments, the one or more stages of the seed train process are implemented automatically or semi-automatically. For example, an automation module, such as seed train process automation module 188 described herein including at least with respect to FIG. 1B, may be used to control an automation system configured to implement one or more stages of the seed train process. The automation may include any suitable components for automating one or more stages of the seed train process. For example, the automation system may include an environmental control system configured to control the environment (e.g., temperature, gas, pressure, pH, etc.) in which the culture of cells is being grown, an imaging system, one or more robotic components configured to administer fluids (e.g., culture media), and/or any other suitable components for automatically or semi-automatically implementing one or more stages of the seed train process.

In some embodiments, real-time culture growth data 142 is generated during implementation of the one or more stages of the seed train process. In some embodiments, the real-time culture growth data 142 includes data that may affect one or more subsequent stages of the seed train process and/or one or more subsequent seed train processes. For example, the data 142 may include data indicative (e.g., specifying or otherwise indicating) of the duration of the stage(s) of the seed train process, the cell doubling time, the final viable cell density, and/or any other suitable data, as aspects of the technology described herein are not limited in this respect. In some embodiments, the real-time culture growth data 142 may be used to update the constraints 102 and/or may be used to determine the seed train process parameters for subsequent stages of the seed train process.

In some embodiments, after implementing the one or more stages of the seed train process at act 140, technique 100 proceeds to act 145 for determining whether an outcome of implementing the stage(s) of the seed train process satisfies one or more success criteria. For example, this may include determining whether the duration of growing the cells during the particular stage(s) exceeds a threshold duration. When the duration exceeds the threshold, this may introduce delays that cause the overall duration of the seed train process to exceed the time allotted for it. Accordingly, technique 100 may end when the duration exceeds the threshold duration, otherwise technique 100 proceeds to act 150. As another example, determining whether an outcome of implementing the stage(s) of the seed train process satisfies one or more success criteria includes determining whether a final viable cell density exceeds a threshold viable cell. When the final viable cell density exceeds the threshold, this may indicate that there is cell overgrowth, which can cause cell death and decrease the quality of the culture. Accordingly, technique 100 may end when the final viable cell density exceeds the threshold, otherwise technique 100 proceeds to act 150.

Act 150 includes determining whether there are additional stages of the seed train process. If there are no additional stages, and the seed train process is complete, then technique 100 ends. If the seed train process is incomplete, meaning there are stage(s) of the seed train process that have not yet been implemented, then the seed train process may be continued.

In some embodiments, if the seed train process is to be continued, the technique 100 returns to act 110 to determine updated seed train process parameters for subsequent stages of the seed train process. For example, the seed train process parameters may be updated to account for the real-time culture growth data 142. In some embodiments, after determining the updated seed train process parameters one or more of acts 120, 130, 140, of technique 100 may be repeated based on the updated seed train process parameters.

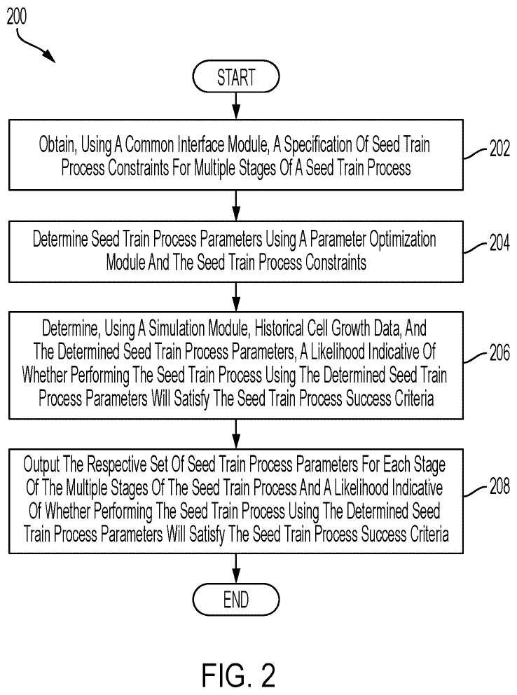

FIG. 1B is a block diagram of an example system 160 for determining seed train process parameters for a seed train process, according to some embodiments of the technology described herein. System 160 includes computing device 170 that is configured to have software 180 execute thereon to perform various functions in connection with designing and/or implementing one or more stages of a seed train process. In some embodiments, software 180 includes a plurality of modules. A module may include processor-executable instructions that, when executed by at least one computer hardware processor, cause the at least one computer hardware processor to perform the function(s) of the module. Such modules are sometimes referred to herein as “software modules.” each of which includes processor executable instructions configured to perform one or more processes, such as the processes described herein including at least with respect to FIGS. 2, 3A, and 4.

The computing device 170 can be one or multiple computing devices of any suitable type. For example, the computing device 170 may be operated by one or more user(s) 176, such as one or more individual(s) who are designing and/or implementing a seed train process. Additionally, or alternatively, the user(s) 176 may include one or more individual(s) associated with the facility where a seed train process is performed. For example, the user(s) 176 may provide, as input to the computing device 170 (e.g., by uploading one or more files), seed train process constraints, initial seed train process parameters, seed train process success criteria, historical cell growth data, and/or any other suitable input as aspects of the technology described herein are not limited to any particular type of input. Additionally, or alternatively, in some embodiments, the user(s) 176 may provide user input specifying processing or other methods to be performed on the seed train process constraints, initial seed train process parameters, seed train process success criteria, historical cell growth data, and/or other suitable data.

As shown in FIG. 1B, software 180 includes multiple software modules for designing and/or implementing a seed train process, such as a parameter optimization module 182, a simulation module 184, a common interface module 186, and a seed train process automation module 188.

In some embodiments, the common interface module 186 is configured to generate a graphical user interface (GUI) to display to user(s) 176, seed train process constraints, seed train process parameters, estimated outcome(s) of the seed train process, an indication as to whether the estimated outcome(s) satisfy seed train process success criteria, and/or any other suitable information, as aspects of the technology are described herein are not limited to displaying any particular type of information. In some embodiments, the common interface module 186 enables multiple different user(s) 176 to view, enter, and/or update such information in a consistent manner. Examples of a display by the common interface module 186 are shown in FIG. 5A-1-FIG. 5D.

In some embodiments, user(s) 176 can provide user input via a GUI (e.g., generated by the common interface module 186) to update one or more seed train process constraints and/or seed train process parameters. For example, user(s) 176 may provide input to update a constraint reflecting a change in the schedule of the facility, thereby allowing a longer or shorter duration for performing one or more stages of the seed train process. Additionally, or alternatively, the user(s) 176 may provide input indicating initial and/or preferred seed train process parameters (e.g., prior to or instead of determining seed train process parameters). In some embodiments, the common interface module 186 automatically updates the display to reflect user changes. For example, the common interface module 186 may automatically generate a GUI to display the updated constraint and/or parameter, as well as the updated estimated outcome(s) resulting from those changes.

In some embodiments, the parameter optimization module 182 is configured to determine seed train process parameters for performing one or more stages of a seed train process. For example, this may include determining, for each particular stage of the seed train process, a respective set of seed train process parameters. Example seed train process parameters are listed in Table 2. In some embodiments, the parameter optimization module 182 is configured to determine the seed train process parameters automatically, or in response to an instruction from the user(s) 176 (e.g., by providing input through a GUI generated by the common interface module 186).

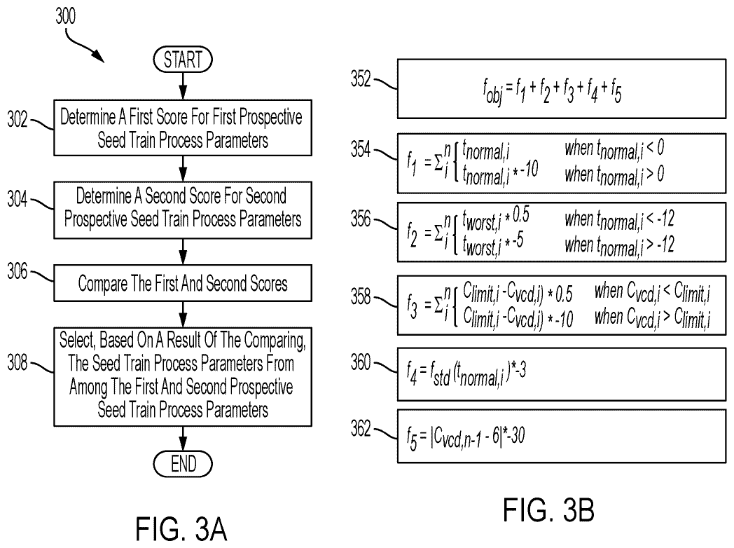

In some embodiments, the parameter optimization module 182 determines seed train process parameters by performing act 110 in FIG. 1A, act 204 in FIG. 2, and/or one or more acts of process 300 described herein including at least with respect to FIG. 3A. For example, in some embodiments, the parameter optimization module 182 uses a genetic algorithm to determine the seed train process parameters. For example, the genetic algorithm may test candidate seed train process parameters against an objective function, such as the objective function in FIG. 3B, to determine the seed train process parameters.

In some embodiments, the parameter optimization module 182 is configured to determine the seed train process parameters using seed train process constraints. As described herein, in some embodiments, seed train process constraints are aspects of the seed train process that cannot be varied. The seed train process constraints may define ranges from which the seed train process parameters should be selected (e.g., initial viable cell density upper and lower limits). Additionally, or alternatively, the seed train process constraints may be used to define seed train process success criteria (e.g., the time allotted for performing a stage of the seed train process). Examples of seed train process constraints are listed in Table 2.

The parameter optimization module 182 may obtain (e.g., pull or receive) seed train process constraints from the seed train process constraint data store 174 and/or the user(s) 176 (e.g., by the user(s) uploading the seed train process constraints). For example, user(s) 176 may upload seed train process constraints using common interface module 186.

In some embodiments, parameter optimization module 182 is additionally, or alternatively, configured to determine seed train process parameters using historical cell growth data. For example, historical cell growth data may include cell doubling time data from one or more prior seed trains. The cell doubling time data may include cell doubling times for each of one or more stages of prior seed train processes(es). In some embodiments, the average and standard deviation of the doubling time at each stage is calculated from the data. In some embodiments, the parameter optimization module 182 may assume an average doubling time for each stage of the seed train process in determining the seed train process parameters.

The parameter optimization module 182 may obtain (e.g., pull or receive) average doubling times from the historical cell growth data store 172 and/or the user(s) 176 (e.g., by the user(s) uploading the historical cell growth data).

In some embodiments, common interface module 186 is configured to generate a GUI to display the parameters determined using the parameter optimization module 182. For example, the common interface module 186 may populate a display with the determined parameters, or may replace initial seed train process parameters, in an existing display, with the determined parameters. The GUI may additionally, or alternatively, display estimated outcome(s) (e.g., duration, final viable cell density, etc.) of implementing one or more stages of the seed train process using the determined parameters. The GUI may additionally, or alternatively, display an indication of whether the estimated outcome(s) satisfy one or more seed train process success criteria. In some embodiments, the user(s) 176 may interact with the GUI to update the seed train process parameter(s) even after they have been determined using the parameter optimization module 182. This may allow user(s) 176 flexibility in designing the seed train process according to user preferences or expectations.

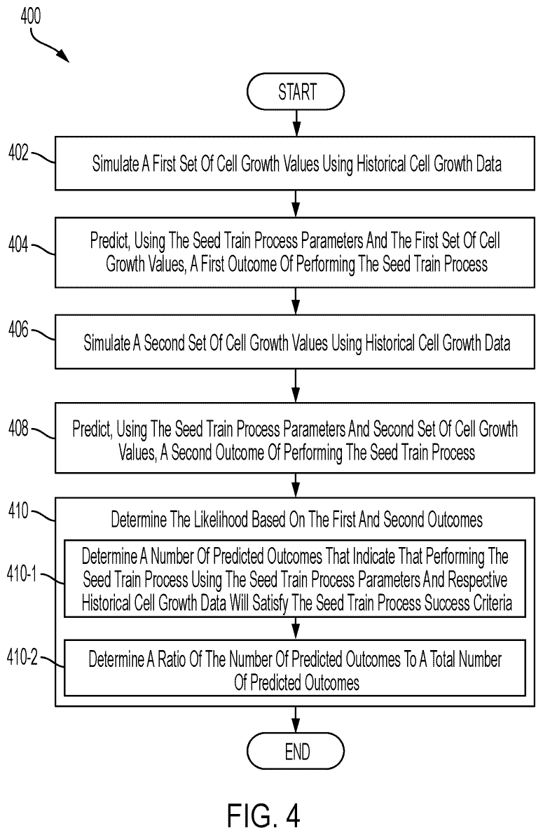

In some embodiments, the simulation module 184 is configured to determine a likelihood indicative of whether performing the seed train process using the seed train process parameters determined using the parameter optimization module 182 will satisfy at least one seed train process success criteria. This may include performing act 120 in FIG. 1A, act 206 in FIG. 2, and/or one or more acts of process 400 described herein including at least with respect to FIG. 4. For example, the simulation module 184 may be configured to evaluate multiple input scenarios using Monte Carlo simulation(s). In some embodiments, as described herein, the Monte Carlo simulations are used to predict whether a particular input scenario will yield outcome(s) that satisfies one or more success criteria. If the outcome(s) are predicted to satisfy at least a threshold number of success criteria (e.g., all success criteria for all stages of the seed train process), then the input scenario may be considered a “successful scenario.” In some embodiments, the likelihood of satisfying the seed train process success criteria is the ratio of the number of successful scenarios to the total number of input scenarios evaluated.

In some embodiments, the simulation module 184 is configured to use historical cell growth data to determine the likelihood of satisfying the seed train process success criteria. For example, an input scenario evaluated by the simulation module 184 may include a set of doubling times randomly sampled from normal distributions calculated from the historical cell data. For example, the set of doubling times may include a doubling time for each stage of the seed train process.

The simulation module 184 may obtain (e.g., pull or receive) historical cell growth data from the historical cell growth data store 172 and/or the user(s) 176 (e.g., by the user(s) uploading the historical cell growth data). For example, user(s) 176 may upload historical cell growth data using common interface module 186.

In some embodiments, the common interface module 186 is configured to output the likelihood of satisfying the seed train process success criteria as determined by the simulation module 184. For example, the common interface module 186 may output information indicative of the likelihood, such as a ratio, percentage, graphic, or any other suitable information indicative of the likelihood. Additionally, or alternatively, in some embodiments, the common interface module is configured to output a likelihood of satisfying the success criteria for individual stage(s) of the seed train process.

In some embodiments, user(s) 176 may determine, based on the output likelihoods of satisfying the success criteria, whether to implement one or more stages of the seed train process. If the user(s) 176 determine to implement one or more stages of the seed train process, in some embodiments, they may instruct seed train process automation module 188 to implement the one or more stages automatically or semi-automatically. Additionally, or alternatively, the user(s) 176 may manually implement the one or more stages of the seed train process.

In some embodiments, the seed train process automation module 188 receives from the simulation module 184, the likelihood of satisfying the seed train process success criteria. The seed train process automation module 188 may be configured to determine, based on the received likelihood, whether to implement one or more stage(s) of the seed train process. For example, if the likelihood equals or exceeds a threshold (i.e., there is a relatively high likelihood of satisfying the seed train process success criteria using the determined seed train process parameters), the seed train process automation module 188 may determine to implement the one or more stages of the seed train process. By contrast, if the likelihood does not equal or exceed the threshold (i.e., there is a relatively low likelihood of satisfying the seed train process success criteria using the determined seed train process parameters), the seed train process automation module 188 may determine not to implement the one or more stages of the seed train process.

In some embodiments, the seed train process automation module 188 may further be configured to control a seed train process automation system 190. For example, the seed train automation module may control the seed train process automation system 190 to implement one or more stages of the seed train process. In some embodiments, seed train process automation system 190 includes any suitable components for automating one or more stages of the seed train process. For example, the automation system 190 may include an environmental control system configured to control the environment (e.g., temperature, gas, pressure, pH, etc.) in which the culture of cells is being grown, an imaging system, one or more robotic components configured to administer fluids (e.g., culture media), and/or any other suitable components for automatically or semi-automatically implementing one or more stages of the seed train process.

Common interface module 186 may be configured to generate a graphical user interface (GUI), a text-based user interface, and/or any other suitable type of interface through which a user may provide input and view information generated by software 180. For example, in some embodiments, the common interface may be a webpage or web application accessible through an Internet browser. In some embodiments, the user interface may be a graphical user interface (GUI) of an app executing on the user's mobile device. In some embodiments, the user interface may include a number of selectable elements through which a user may interact. For example, the user interface may include dropdown lists, checkboxes, text fields, or any other suitable element.

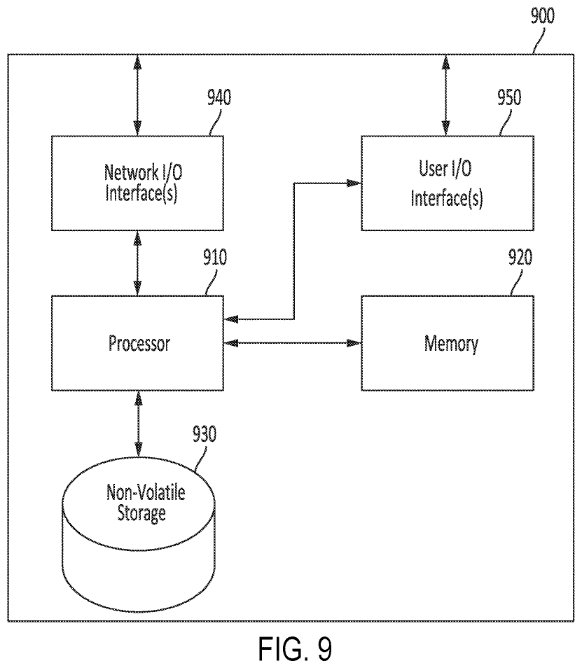

FIG. 2 is a flowchart of an illustrative process 200 for determining seed train process parameters for a seed train process having multiple stages, according to some embodiments of the technology described herein. One or more acts of processes 200 may be performed automatically by any suitable computing device(s). For example, the act(s) may be performed by a laptop computer, a desktop computer, one or more servers, in a cloud computing environment, computing device 900 as described herein within respect to FIG. 9, and/or in any other suitable way. For example, in some embodiments, act 202 may be performed automatically may be performed automatically by any suitable computing device(s). As another example, act 204 may be performed automatically by any suitable computing device(s).

At act 202, the processor obtains a specification of seed train process constraints for multiple stages of a seed train process. As described herein, seed train process constraints are aspects of the seed train process that cannot be varied. For example, the seed train process constraints may include at least some of the seed train process constraints listed in Table 1 and/or the seed train process constraints 102 described herein including at least with respect to FIG. 1A.

In some embodiments, the seed train process constraints include one or more constraints for each of multiple stages of the seed train process. For example, first seed train process constraints may be specified for a first stage in the seed train process and second seed train process constraints may be specified for a second stage in the seed train process. In some embodiments, different seed train process constraints are specified for different stages (e.g., the first and the second stages). For example, some seed train process constraints, such as the upper and lower bounds of the working volume, may depend on the volume of container(s) used for growing a culture of cells during a particular stage. If the volume of containers increases with each stage, the upper and lower bounds of the working volume range will also increase. FIG. 5A-1-FIG. 5C-2 show example seed train process constraints specified for each of stages N−8 through N−0 of an example seed train process.

In some embodiments, the processor obtains the specification of seed train process constraints using a common interface module. For example, the common interface module may include common interface module 186 described herein including at least with respect to FIG. 1B. In some embodiments, the common interface module indicates relevant seed train process constraints and include fields that store values for each of the seed train process constraints. User(s) may interact with a GUI generated by the common interface module to provide and/or update values for the seed train process constraints (e.g., using text boxes or other selectable elements of a graphical user interface). As an example, when a seed train process is implemented at a facility where multiple other processes (e.g., other seed train processes) are implemented, users of the facility may interact with the GUI to update facility-related constraints, such as available equipment (e.g., containers), scheduling constraints, and any other suitable constraints, as aspects of the technology are not limited in this respect. Accordingly, by obtaining seed train process constraints through the common interface module, the processor can obtain up-to-date seed train process constraints, which can be used to design a seed train process that complies with facility scheduling. Additionally, or alternatively, the processor obtains the specification of seed train process constraints from a data store (e.g., seed train process constraint data store 174 in FIG. 1B), or using any other suitable techniques, as aspects of the technology described herein are not limited in this respect.

At act 204, the processor determines seed train process parameters using the seed train process constraints. As described herein, seed train process parameters are aspects of the seed train process that can be varied. For example, the seed train process parameters may include at least some of the seed train process parameters listed in Table 2 and/or the seed train process parameters described herein including at least with respect to FIG. 1A.

In some embodiments, the processor determines seed train process parameters for each particular stage of the multiple stages of the seed train process, a respective set of seed train process parameters for growing the culture during the particular stage. For example, this may include determining a first set of seed train process parameters for a first stage in the seed train process and a second set of seed train process parameters for a second stage in the seed train process. FIG. 5B-1-FIG. 5C-2 show example seed train process parameters determined for each of stages N−8 through N−0.

In some embodiments, the processor determines the seed train process parameters using a parameter optimization module, such as parameter optimization module 182 described herein including at least with respect to FIG. 1B.

In some embodiments, determining the seed train process parameters includes determining the seed train process parameters using an optimization technique. Any suitable optimization technique may be used, including, for example, a genetic algorithm, a random search, or a grid search, as embodiments of the technology described herein are not limited in this respect. In some embodiments, a genetic algorithm may take, as input, an objective function and bounds for each variable in the function. For example, the bounds may be defined by seed train process constraints, and the objective function may include the example objective function in FIG. 3B. In some embodiments, the genetic algorithm generates a set of values for variables in the objective function, randomly chosen from the ranges defined by the specified bounds, that mutate through iterations until a set of values is found that render a global minimum (or maximum). In some embodiments, a genetic algorithm is a differential evolution algorithm. For example, Storn, R. and Price, K. (Differential Evolution—A simple and efficient adaptive scheme for global optimization over continuous spaces), which is incorporated by reference herein in its entirety, describes an example differential evolution algorithm. Example techniques for determining seed train process parameters are described herein including at least with respect to FIG. 3A.

In some embodiments, in determining the seed train processor parameters, the processor may estimate one or more outcomes of implementing one or more stages of the seed train process using a prospective set of parameters and the specified constrained. For example, this may include estimating outcome(s) of implementing a first stage of the seed train process using a first set of prospective parameters for that stage and the constraints that were specified for the first stage. In some embodiments, the estimated outcomes include an estimated duration needed to grow the culture of cells during a particular stage of the seed train process and/or an estimate final viable cell density resulting from growing the culture of cells during the particular stage.

In some embodiments, the seed train process parameters are determined at act 204 such that one or more (e.g., one, some, or all) of the estimated outcomes of implementing the one or more stages of the seed train process satisfy seed train process success criteria. As described herein, including at least with respect to FIG. 1A, the success criteria may include: (a) a criterion that an expected duration needed to grow the culture during a particular stage does not exceed a threshold duration for growing the culture during the particular stage, and/or (b) a criterion that an expected viable cell density resulting from growing the culture during a particular stage of the seed train process does not exceed a threshold viable cell density.

In some embodiments, when multiple prospective sets of parameters result in outcomes that satisfy the success criteria, only one set of the prospective parameters may be identified as the seed train process parameters. For example, when testing prospective seed train process parameters against an objective function, the processor may select the parameters that result in a value of the objective function that meets at least one criterion. For example, the prospective parameters that result in the largest or the smallest value of the objective function may be selected as the seed train process parameters.

At act 206, the processor determines a likelihood indicative of whether performing the seed train process using the determined seed train process parameters will satisfy the seed train process success criteria. In some embodiments, the processor determines the likelihood using a simulation module, historical cell growth data, and the determined seed train process parameters. The simulation module may include simulation module 184 in FIG. 1B.

In some embodiments, as described herein, the historical cell growth data includes data related to the growth rate of cells from past seed train experiments. For example, the historical cell growth data may include culture doubling time data from past seed train processes. The doubling time data may include a distribution of doubling time values for each of multiple stages of past seed train processes.

Since, in some embodiments, the outcomes of performing a seed train process using the determined seed train process parameters are estimated based on only one or a few doubling times (e.g., an average historical doubling time, a shortest historical doubling time, and/or a longest historical doubling time), the estimated outcomes do not account for other potential doubling times (e.g., those included in the distributions of doubling times). Accordingly, the estimated outcomes may not accurately reflect the actual outcomes of performing the seed train process. For example, growing a culture during a particular stage of the seed train process take longer than an estimate duration when the estimate duration is estimated based on the historical average doubling time and the actual doubling time is longer than the historical average. In this example, while the estimate duration may satisfy the success criteria, the actual duration may not satisfy the success criteria.

Accordingly, in some embodiments, it may be beneficial to determine the likelihood of satisfying the success criteria prior to implementing one or more stages of the seed train process using the determined seed train process parameters. If the likelihood of satisfying the success criteria is relatively low, this may indicate that implementing the seed train process using the determined seed train process parameters is likely to violate the success criteria (e.g., it may result in cell overgrowth or take longer than the time allotted for implementing a particular stage).

In some embodiments, determining the likelihood of satisfying the seed train process success criteria includes (a) sampling historical cell growth data, (b) simulating the outcomes of performing the seed train process using the sampled historical cell growth data and the seed train process parameters determined at act 204, and (c) determining whether the simulated outcomes satisfy the success criteria. Techniques for determining the likelihood of satisfying the success criteria are described herein including at least with respect to FIG. 4.

At act 208, the processor outputs (a) the set of seed train process parameters determined for each stage of the multiple stages of the seed train process, and (b) the likelihood indicative of whether performing the seed train process using the determined seed train parameters will satisfy the seed train process success criteria.

In some embodiments, the processor outputs the sets of seed train process parameters and/or the likelihoods via a common interface module. For example, FIG. 5B-1-FIG. 5D show a set of seed train process parameters determined for each of the stages N−8 through N−1. In some embodiments, the parameters and/or likelihood are displayed via a user interface, such as a GUI generated by the common interface module 186 in FIG. 1B. Additionally, or alternatively, in some embodiments, the parameters and/or likelihood are output (e.g., transmitted) to a seed train process automation module, such as seed train process automation system 190 in FIG. 1B. Additionally, or alternatively, in some embodiments, the parameters and/or likelihood are output to a data store, or output using any other suitable output technique, as aspects of the technology described herein are not limited in this respect.

In some embodiments, the likelihood indicative of whether performing the seed train process using the determined seed train process parameters will satisfy the success criteria is used to determine whether to implement one or more stages of the seed train process. For example, this may include determining to implement the one or more stages of the seed train process when the output likelihood exceeds a threshold likelihood. In some embodiments, a user, such as a scientist, manually determines whether to implement the one or more stages of the seed train process. In some embodiments, the decision to implement the one or more stages is made automatically or semi-automatically using a processor. For example, the seed train process automation module 188 in FIG. 1B may automatically determine whether to implement the one or more stages of the seed train process.

Though not show, in some embodiments, process 200 may include implementing one or more stages of the seed train process using the determined seed train process parameters. For example, the one or more stages of the seed train process may be implemented manually (e.g., by a user), automatically, or semi-automatically. For example, a seed train process automatization system, such as seed train process automation system 190 in FIG. 1B, may automatically or semi-automatically implement the one or more stages of the seed train process.

It should be appreciated that process 200 may include one or more additional or alternative acts not shown in FIG. 2A. For example, process 200 may include an act for determining whether to implement one or more stages of a seed train process and/or an act for implementing the one or more stages of the seed train process.

FIG. 3A is a flowchart of an illustrative process 300 for determining the seed train process parameters using the parameter optimization module and the seed train process constraints, according to some embodiments of the technology described herein. In some embodiments, process 300 is an example implementation of act 204 of process 200 in FIG. 2.

One or more acts of processes 300 may be performed automatically by any suitable computing device(s). For example, the act(s) may be performed by a laptop computer, a desktop computer, one or more servers, in a cloud computing environment, computing device 900 as described herein within respect to FIG. 9, and/or in any other suitable way. In some embodiments, a software module, such as parameter optimization module 182 in FIG. 1B, is configured to perform process 300.

In some embodiments, process 300 includes evaluating multiple prospective sets of seed train process parameters to identify seed train process parameters that, when used to implement a seed train process, will result in an outcome of the seed train process that satisfies seed train process success criteria. For example, the multiple prospective sets of parameters may include any suitable number of prospective sets of parameters, including first prospective parameters and second prospective parameters. The first prospective parameters and the second parameters may not include any of the same parameters or may include one or more of the same parameters.

In some embodiments, a prospective set of parameters includes prospective seed train process parameters for each stage in the seed train process. In some embodiments, each prospective parameter for a particular stage in the seed train process is selected to comply with the seed train process constraints for that stage. For example, a prospective working volume for a first stage of the seed train process may be selected such that it is within (or equivalent to) the upper working volume limit and the lower working volume limit for the first stage, both of which are seed train process constraint for the first stage.

At act 302, the processor determines a first score for the first prospective seed train process parameter. For example, determining a first score may include using the first prospective seed train process parameters to determine a value for an objective function, such as the objective shown in FIG. 3B. In some embodiments, as described in more detail herein, the objective function accounts for each of the success criteria. Accordingly, in some embodiments, the resulting value of the objective function reflects whether using the first prospective parameters for implementing one or more stages of the seed train process will result in outcomes that satisfy the success criteria for each stage. Additionally, or alternatively, in some embodiments, the resulting value of the objective function reflects the degree to which the success criteria will be satisfied. For example, a relatively large value (or a relatively small value) of the objective function may indicate that using the first prospective parameters to implement one or more stages of the seed train process will result in outcomes that satisfy the success criteria to a greater degree.

In some embodiments, determining the degree to which the estimated outcomes satisfy the seed train process success criteria includes determining a number of estimated outcomes that satisfy the seed train process success criteria. For example, multiple outcomes may be estimated for each stage in the seed train process. In some embodiments, the value of the objective function is larger (or smaller) when more of the estimated outcomes satisfy the success criteria at each stage. For example, the value of the objective function may be larger when the estimated outcomes satisfy all criteria for each stage, as compared to the value of the objective function when the estimated outcomes satisfy only some of the success criteria for one or more stages.

Additionally, or alternatively, in some embodiments, determining the degree to which the estimated outcomes satisfy the seed train process criteria includes determining the degree to which each individual estimated outcome satisfies a respective success criterion. Consider, for example, an estimate duration of 5 hours for growing cells during the first stage of the seed train process and a threshold value of 10 hours. In the example, the success criterion is satisfied when the estimate duration does not exceed the threshold. Here, the success criterion is satisfied since the estimate duration of 5 hours does not exceed 10 hours. The estimate duration of 5 hours also satisfies the success criterion to a greater degree than an estimate duration of 8 hours since the estimate duration of 8 hours is closer to the threshold duration.

At act 304, a second score is determined for second prospective seed train process parameters. As described with respect to act 302, determining a score for prospective seed train process parameters, in some embodiments, includes determining a value of an objective function, such as the objective function shown in FIG. 3B.

At act 306, the processor compares the first score determined for the first prospective parameters and the second score determined for the second prospective parameters. For example, this may include determining the relative value of the first score with respect to the second score.

At act 308, based on a result of the comparing, the processor selects the seed train process parameters from among the first prospective parameters and the second prospective parameters. In some embodiments, this includes selecting the set of prospective parameters for which the highest score (or lowest score) was determined. For example, if the first score is determined to be larger than the second score at act 306, then the first prospective parameters may be selected as the seed train process parameters. In some embodiments, a higher (or lower) score may indicate that using the prospective parameters, for which that score was determined, to implement a stage of the seed train process, will result in an outcome that satisfies the success criteria to a greater degree.

It should be appreciated that one or more additional prospective sets of parameters may be evaluated during process 300, and that process 300 is not limited to evaluating only first prospective parameters and second prospective parameters. Rather, any suitable number of sets of prospective parameters may be evaluated and used to determine the seed train process parameters. For example, process 300 may include determining a third score for third prospective parameters, comparing the first, second, and third scores, and selecting the seed train process parameters from among the first, second, and third prospective parameters. Additionally, or alternatively, the processor may search prospective seed train process parameters within the seed train process constraints until a combination of prospective seed train process parameters is found that results in a threshold value of the objective function.

FIG. 3B shows an example objective function used for determining seed train process parameters, according to some embodiments of the technology described herein. In some embodiments, the objective function is used to determine seed train process parameters as part of process 300 in FIG. 3A. For example, the seed train process parameters may be searched to identify a combination of seed train process parameters that maximize equation 352.

As shown, the example objective function, f_{obj}, 352 may be calculated as a summation of functions f₁, f₂, f₃, f₄, and f₅.

In some embodiments, the function, f₁, 354 accounts for at least one success criterion. For example, the function 354 may account for a success criterion that the extra time needed to grow cells during each stage (e.g., stages 1 through n) of the seed train process is lower than a threshold time for that particular stage, where the extra time needed to grow the cells is based on the normal doubling time for the stage. As described herein, the normal doubling time for a particular stage may be the average doubling time for the stage based on historical cell growth data. n may be equal to the total number of stages in the seed train process. Additionally, or alternatively, n may be less than the total number of stages in the seed train process. For example, FIG. 5A-1-FIG. 5D show that stages N−3 and N−2 each use the same container. In this example, the two stages may only count as a single stage, as opposed to two different stages.

With respect to function 354, t_{normal,1} refers to the extra time that is needed to grow the culture at a stage, i, of the seed train process, where the extra time is determined based on a normal doubling time.

As shown, function 354 is determined by comparing the extra time needed at each stage (e.g., stages 1 through n) of the seed train process to a threshold extra time. In this case, the threshold extra time is 0. If the extra time needed exceeds the threshold time for a particular stage, then the summand for that particular stage is calculated by multiplying t_{normal,i} by −10. If the extra time needed is below the threshold, then the summand for the particular stage is t_{normal,i}.

In some embodiments, the function 356 accounts for at least one other success criterion. For example, the function 356 may account for a success criterion that the extra time needed to grow cells during each stage (e.g., stages 1 through n) of the seed train process is lower than a threshold time for that particular stage, where the extra time needed to grow the cells is based on the worst doubling time for the stage. As described herein, the worst doubling time for a particular stage may be the longest doubling time that is included in historical cell growth data for that stage.

With respect to function 356, t_{worst,1} refers to the extra time that is needed to grow the culture at a stage, i, of the seed train process, where the extra time is determined based on a worst doubling time for that stage.

As shown, function 356 is determined by comparing the normal extra time needed t_{normal,i} at each stage (e.g., stages 1 through n) of the seed train process to a threshold extra time. In this case, the threshold extra time is −12. If the extra time needed exceeds the threshold time for a particular stage, then the summand for that particular stage is calculated by multiplying t_{worst,i} by 0.5. If the extra time needed is below the threshold, then the summand for the particular stage is t_{worst,i} multiplied by −5.

In some embodiments, the function 358 accounts for at least one other success criterion. For example, the function 358 may account for a success criterion that the final viable cell density resulting from growing the cells during each stage (e.g., stages 1 through n) of the seed train process is lower than a threshold final viable cell density.

With respect to function 358, C_{vcd,i} refers to the final viable cell density resulting from growing cells during a stage, i, of the seed train process. In some embodiments, the final viable cell density for each stage is calculated based on the best doubling time for the stage. For example, the best doubling time may refer to the shortest doubling time for the stage as obtained from historical cell growth data. C_{limit,i} refers to the threshold viable cell density for the particular stage. For example, the threshold viable cell density may include the upper limit of the final viable cell density for a particular stage of the seed train process, which is specified as part of obtaining seed train process constraints for the seed train process.

As shown, function 358 is determined by comparing the final viable cell density, C_{vcd,i}, to the threshold viable cell density, C_{limit,i}, for each stage (e.g., stages 1 through n) of the seed train process. If C_{vcd,i} is less than C_{limit,i}, then the summand for that particular stage is calculated by multiplying the difference between C_{limit,i} and C_{vcd,i} by 0.5. If C_{vcd,i} exceeds C_{limit,i}, then the summand for that particular stage is calculated by multiplying the difference between C_{limit,i} and C_{vcd,i} by −10.

In some embodiments, the function 360 accounts for a safety margin for completing the seed train process within the time constraints for completing the seed train process. For example, if one stage has unexpected slow cell growth, the seed train process parameters should allow for enough of a safety margin for completing the seed train process within the allotted time. Having enough of a safety margin with respect to the time for growing cells during the next stage may assist in keeping the duration of the seed train process in compliance with facility scheduling and/or the allotted time for completing the seed train process. In some embodiments, this is done by balancing the extra time needed for growing the cells during each stage.

In some embodiments, the function 360 is determined by multiplying f_{std}(t_{normal,i}) by −3, where f_{std}(t_{normal,i}) is a function for determining the standard deviation of the extra time needed (t_{normai,i}) for different stages of the seed train process, where the extra time needed is based on the normal doubling time for each stage.

In some embodiments, the function 362 accounts for variations in the initial viable cell density. For example, it may not be desirable to vary from a recommended value of the initial viable cell density.

In some embodiments, the function 362 is determined based on the final viable cell density, C_{vcd,n-1}, for a preceding stage, n−1, of the seed train process, where C_{vcd,n-1} is determined based on the normal doubling time for stage n−1. For example, the function 362 may be calculated by multiplying the absolute value of the difference between C_{vcd,n-1} and 6 by −30.

FIG. 4 is a flowchart of an illustrative process 400 for determining a likelihood indicative of whether performing the seed train process using the seed train process parameters will satisfy seed train process success criteria, according to some embodiments of the technology described herein. In some embodiments, process 400 is an example implementation of act 206 of process 200 in FIG. 2.

One or more acts of processes 400 may be performed automatically by any suitable computing device(s). For example, the act(s) may be performed by a laptop computer, a desktop computer, one or more servers, in a cloud computing environment, computing device 900 as described herein within respect to FIG. 9, and/or in any other suitable way. In some embodiments, a software module, such as simulation module 184 in FIG. 1B, is configured to perform process 400.

As described herein, in some embodiments, outcomes of the seed train process depend on seed train process constraints and seed train process parameters used to implement each stage of the seed train process. Doubling time is one such seed train process constraint. However, the doubling time varies across different seed train processes. Accordingly, an outcome (e.g., duration of each stage, final viable cell density resulting from each stage, etc.) of the seed train process may depend on the particular doubling time for the culture being grown. Since this value is not known until after implementing one or more of the stages of the seed train process, it can be challenging to predict whether implementing the seed train process using particular seed train process parameters will result in an outcome that satisfies seed train process success criteria. As described herein, the seed train process parameters may be determined assuming a particular value for the doubling time for each stage, such as the average doubling time for each stage of one or more past seed train processes.

Accordingly, it may be beneficial to predict, using process 400, based on historical cell growth data (e.g., historical doubling times), the likelihood that implementing the seed train process using the determined seed train process parameters will result in an outcome that satisfies the success criteria.

At act 402, the processor simulates a first set of cell growth values using historical cell growth data. In some embodiments, the historical cell growth data includes a distribution of historical cell growth values for each stage of the seed train process. As described herein, historical cell growth values for a particular stage of the seed train process may include historical doubling times for that particular stage in the seed train process.

In some embodiments, simulating the first set of historical cell growth values includes randomly sampling the historical cell growth values from the historical cell growth data. This may include, for each stage, randomly sampling a cell growth value from the distribution of cell growth values for that particular stage. For example, simulating the first set of historical cell growth values may include, for a first stage, randomly sampling a doubling time from a distribution of historical doubling times for that stage.

At act 404, the processor predicts a first outcome of performing the seed train process using the seed train process parameters and the first set of cell growth parameters. In some embodiments, this includes predicting a respective outcome for each stage of the seed train process based on the sampled historical cell value (e.g., doubling time) for that stage. For example, predicting an outcome for a particular stage of the seed train process may include predicting a duration for growing the culture during the particular stage. The duration for a particular stage may be predicted using Equation 1, for example.

$$Expected Duration = \frac{ln ( \frac{( \begin{matrix}Minimum Final \\ VCD Required\end{matrix} ) \cdot ( \begin{matrix}( Total Vol . \\ After Loss\end{matrix} ) + 5 )}{( Working Vol Per Vessel ) * ( \# Vessels ) * ( Initial VCD )} )}{Grow𝔱h Constant} \qquad ( Equation 1 )$$

$$where : \\ Growth Constant = \frac{ln ( 2 )}{Sampled Doubling Time} \qquad ( Equation 2 )$$

Additionally, or alternatively, in some embodiments, predicting an outcome for the particular stage includes predicting an amount of extra time that will be needed to grow the culture during the particular stage relative to a standard culture time (e.g., 72 hours). The extra time needed may be predicted using Equation 3, for example.

$$Extra Time Needed = Expected Duration - Standard Culture Time \qquad ( Equation 3 )$$

Additionally, or alternatively, in some embodiments, predicting an outcome for the particular stage includes predicting the final viable cell density resulting from growing the culture during the particular stage. The final viable cell density (VCD) resulting from growing the culture during a particular stage may be predicted using Equation 3, for example.

$$Final VCD = ( Initial VCD ) * {e}^{( ( Normal Growth Constant ) * ( Standard Culture Time ) )} \qquad ( Equation 4 )$$

Where the normal doubling time is determined using Equation 2.

In some embodiments, variables included in Equations 1-4 may correspond to seed train process parameters and/or seed train process constraints listed in Tables 1-2.

At act 406, the processor simulates a second set of cell growth values using historical cell growth data. Techniques for simulating a set of cell growth values are described with respect to act 402. In some embodiments, the first set of cell growth values and the second set of cell growth values are different. For example, the first and second sets of cell growth values may include only some of the same cell growth values or may include none of the same cell growth values.

At act 408, the processor predicts a first outcome of performing the seed train process using the seed train process parameters and the first set of cell growth parameters. In some embodiments, this includes predicting a respective outcome for each stage of the seed train process based on the sampled historical cell value (e.g., doubling time) for that stage. For example, this may include determining an expected duration of each stage, the extra time needed for each stage, and/or the final viable cell density for each stage. Such outcomes may be determined using Equations 1-4.

At act 410, the processor determines the likelihood indicative of whether performing the seed train process using the seed train process parameters will satisfy seed train process success criteria. The likelihood is determined using the first and second outcomes. For example, the first and the second outcomes may each include one or more predicted outcomes for each stage of the seed train process.

In some embodiments, determining the likelihood includes, at 410-1, determining a number of predicted outcomes that indicate that performing the seed train process using the seed train process parameters and respective sets of historical cell growth values will satisfy the seed train process success criteria. In some embodiments, this includes determining whether the first outcome and the second outcome satisfy the seed train process success criteria. Additionally, or alternatively, this includes determining one or more other predicted outcomes satisfy the seed train process success criteria. For example, the process may predict at least 5, at least 10, at least 15, at least 25, at least 50, at least 75, at least 100, at least 150, at least 200, or any other suitable number of outcomes of performing the seed train process, using the determined seed train process parameters and respective sets of historical cell growth values.

As described herein, the first outcome may include one or more predicted outcomes for each of multiple stages of the seed train process. Accordingly, in some embodiments, determining whether the first outcome satisfies the success criteria includes determining whether each of the one or more predicted outcomes satisfies a respective success criterion. For example, the first outcome may include a predicted duration for growing cells during the first stage of the seed train process. The processor may determine whether the predicted duration satisfies a respective success criterion. For example, the processor may determine whether the predicted duration is below a threshold duration, thereby satisfying a first success criterion.

In some embodiments, if at least a threshold proportion (e.g., at least 60%, at least 70%, at least 80%, at least 90%, or 100%) of the predicted outcomes satisfy a respective success criterion, then the first outcome is determined to satisfy the seed train process success criteria. For example, if each of the outcomes predicted for each stage of the seed process satisfy its respective success criterion, then the first outcome is determined to satisfy the seed train process success criteria. In other words, implementing the seed train process using the determined seed train process parameters, when the culture's doubling times are those that are included in the first set of historical cell growth values, are likely to result in outcomes that satisfy the seed train process criteria.

In some embodiments, this is repeated for each predicted outcome, including the second outcomes. For example, the processor may determine whether the second outcome satisfies the success criteria. Determining whether the second outcome satisfies the success criteria may be implemented according to the techniques described above for determining whether the first outcome satisfies the success criteria.

At act 410-2, the processor determines a ratio of the number of predicted outcomes that satisfy the success criteria to a total number of predicted outcomes. For example, if the predicted outcomes include the first outcome and the second outcome, and only the first outcome is determined to satisfy the success criteria, then the processor determines a ratio of ½ or 50%. As another example, if 75 out of 100 predicted outcomes are determined to satisfy the success criteria, then the processor determines a ratio of 75/100 or 75%. In some embodiments, the ratio determined at act 410-2 is the likelihood indicative of whether performing the seed train process using the seed train process parameters will satisfy seed train process success criteria.

FIG. 5A-1-FIG. 5D show an example user interface for specifying and viewing seed train process information. FIG. 5A-1-FIG. 5D further include example seed train process constraints and example seed train process parameters for a 5000 mL seed train process.

As shown in FIG. 5A-1-FIG. 5D, the user interface displays seed train process stage information. The seed train process stage information is indicated by its shading as represented in legend 510. In some embodiments, the seed train process information specifies the stage, the passage number, and the container.

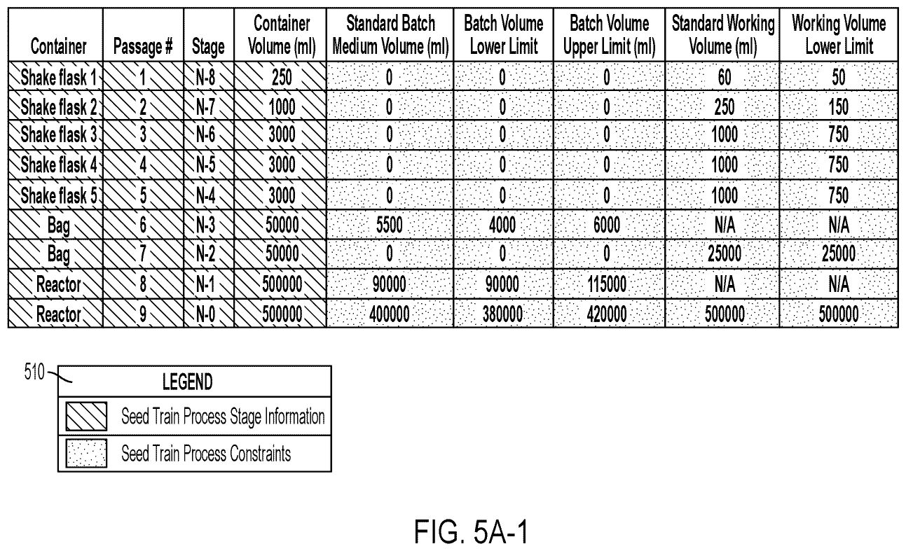

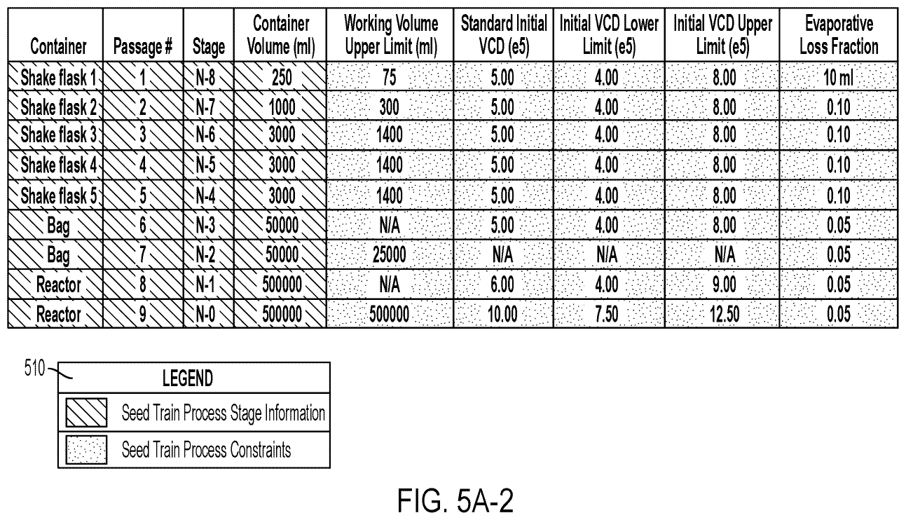

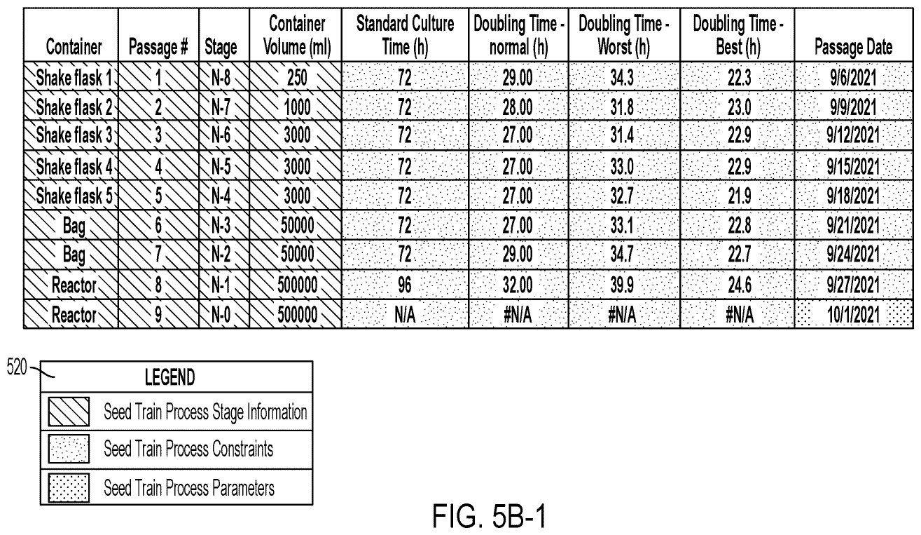

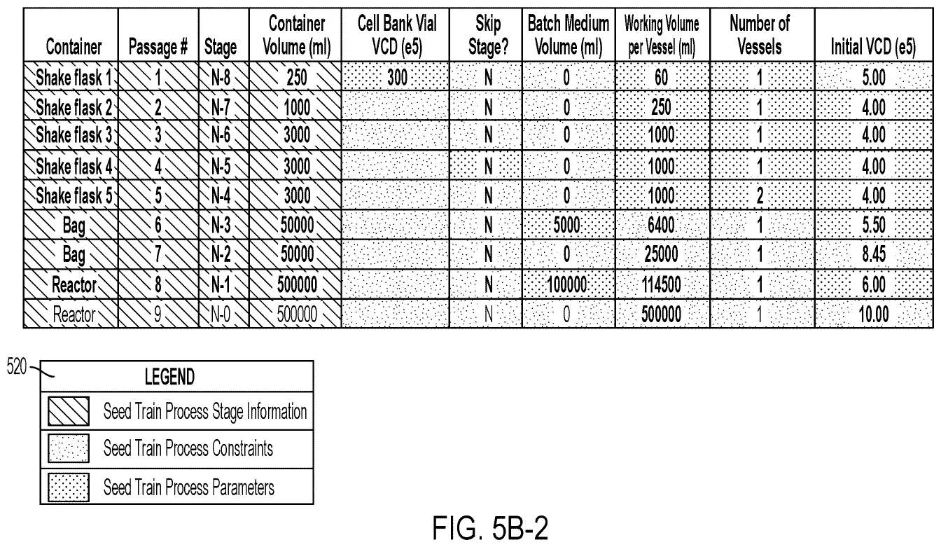

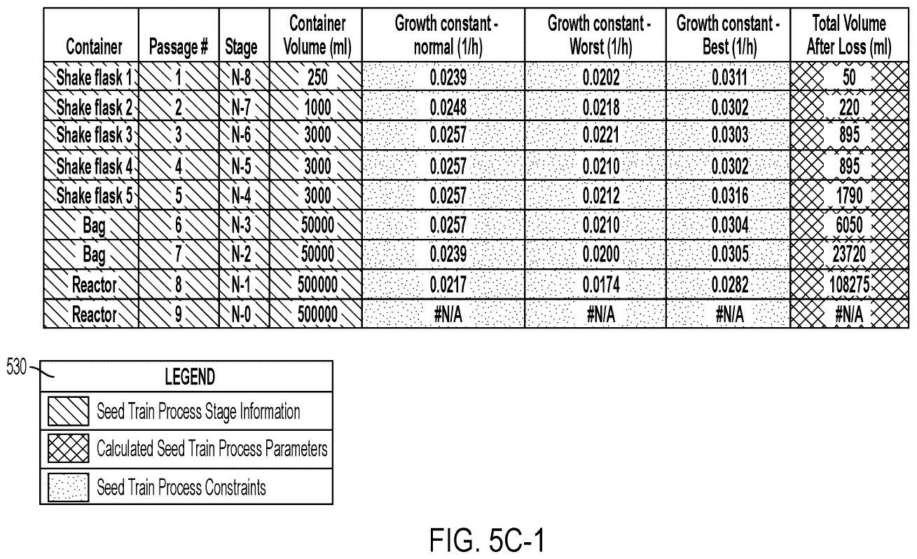

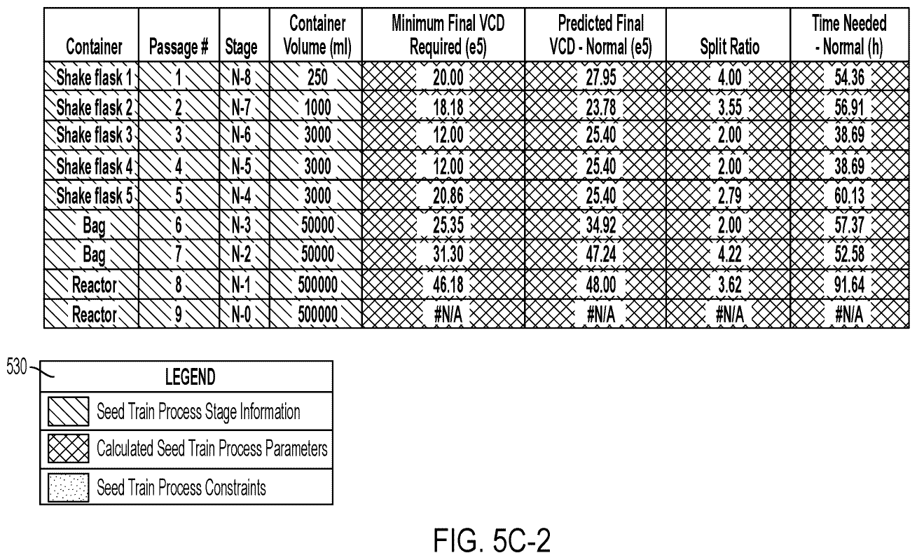

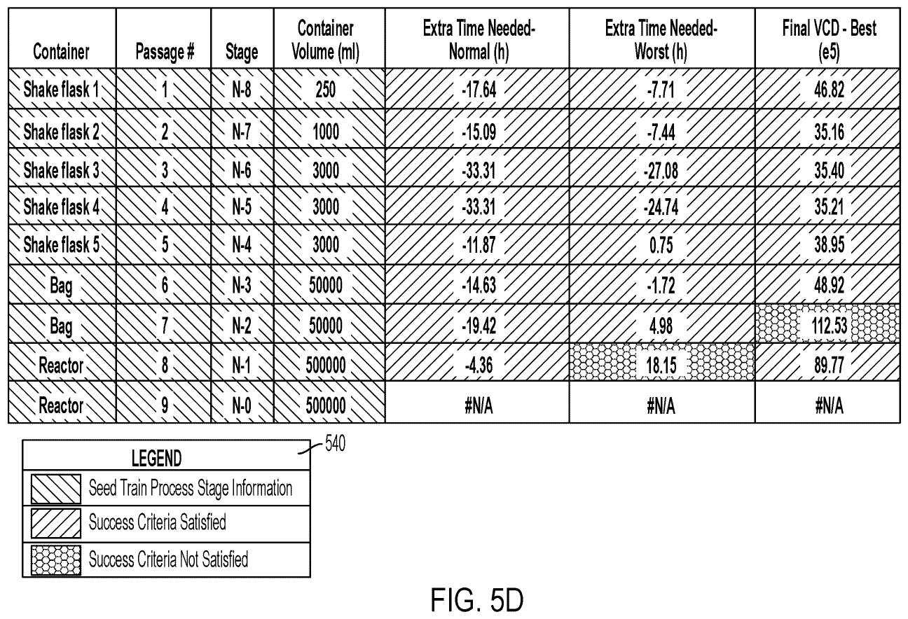

As shown in FIG. 5A-1, FIG. 5A-2, FIG. 5B-1, FIG. 5B-2, FIG. 5C-1, and FIG. 5C-2, the user interface displays seed train process constraints. The seed train process constraints are indicated by their shading as represented in legends 510, 520, and 530. In some embodiments, a user may specify values for seed train process constraints by interacting with the user interface. For example, the text boxes may be selectable elements of a graphical user interface. Additionally, or alternatively, the seed train process constraints may be populated automatically based on stored data and/or data that has been uploaded (e.g., uploaded from another device, such as another computing device, a seed train process automation module, or in any other suitable manner). Example seed train process constraints are shown in FIG. 5A-1-FIG. 5C-2 and may also include any other seed train process constraints described herein including those listed in Table 1.

As shown in FIG. 5B-1 and FIG. 5B-2, the user interface also displays seed train process parameters. The seed train process parameters are indicated by their shading as represented in legend 520. In some embodiments, the seed train process parameters are populated based on a result of determining the seed train process parameters according to the techniques described herein. For example, the seed train process parameters may be determined by performing processes 200 in FIG. 2, process 300 in FIG. 3A, and/or process 400 in FIG. 4. Additionally, or alternatively, a user may specify values for seed train process parameters by interacting with the user interface or obtained in any other suitable manner. Example seed train process parameters are shown in FIG. 5A-1, FIG. 5A-2, FIG. 5B-1, FIG. 5B-2, FIG. 5C-1, and FIG. 5C-2 and may also include any other seed train process parameters described herein including those listed in Table 2.

As shown in FIG. 5A-1, FIG. 5A-2, FIG. 5B-1, FIG. 5B-2, FIG. 5C-1, and FIG. 5C-2, seed train process parameters and seed train process constraints may be specified and/or determined for each stage in the seed train process. Some of the variables are considered to be seed train process constraints for some stages, but seed train process parameters for other stages. For example, the working volume per vessel is a parameter that can be varied for the first five stages, but a seed train process constraint for the remaining four stages.

FIG. 5C-1 and FIG. 5C-2 also show calculated seed train process parameters. In some embodiments, calculated seed train process parameters are those that are calculated based on at least one seed train process parameter (e.g., those that are determined according to the seed train process parameter determination techniques described herein). The calculated seed train process parameters are indicated by the shading shown in legend 530. Example equations for calculating the calculated seed train process parameters are listed in Table 3.

FIG. 5D shows example predictions of whether performing the seed train process using the seed train process parameters will satisfy the seed train process success criteria, according to some embodiments of the technology described herein. At least three columns in FIG. 5D correspond to example predicted outcomes of performing the seed train process using the seed train process parameters and the calculated seed train process parameters. For example, the predicted outcomes include, for each stage, “Extra Time Needed-Normal,” “Extra Time Needed-Worst,” and “Final VCD.” In some embodiments, the “Extra Time Needed-Normal” refers to the extra time needed to grow cells during a particular stage of the seed train process when the culture has an average doubling time. The “Extra Time Needed-Worst” may refer to the extra time need to grow cells during a particular stage of the seed train process when the culture has the longest doubling time determined from historical cell growth data. The “Final VCD-Best” may refer to the viable cell density resulting from growing cells during the particular stage when the culture has the shortest doubling time determined from historical cell growth data. Example equations for determining “Extra Time Needed-Normal,” “Extra Time Needed-Worst,” and “Final VCD-Best” are listed in Table 4.

As described herein, in some embodiments, the predicted outcomes are evaluated to determine whether they satisfy respective success criteria. In some embodiments, the user interface provides an indication as to whether the example outcomes satisfy respective success criteria. As indicated by the legend 540, the shading of the user interface indicates when the success criteria have been satisfied or not. For example, as shown, the “Extra Time Needed-Worst” predicted for stage N−1 and the “Final VCD-Best” predicted for stage N−2 do not satisfy the seed train process success criteria.

In the example, each predicted outcome is compared to a threshold value to determine whether a success criterion is satisfied. For example, the values predicted for “Extra Time Needed-Worst” may be compared to a threshold of 12 hours, the values predicted for “Extra Time Needed-Normal” may be compared to a threshold of 0, the values predicted for “Final VCD—Best” for stages N−1 and N−2 may be compared to a threshold of 100, and the values predicted for “Final VCD—Best” for stages N−8 through N−3 may be compared to a threshold of 50.

In some embodiments, as constraints and parameters are changed, the shading indicating whether the success criteria have been satisfied updates automatically, allowing for a user to easily visualize and estimate the outcome of different stages of the seed train process.

TABLE 3
Equations for determining the calculated seed train parameters.

| Calculated | | |
|---|---|---|
| Seed Train | | |
| Process | | |
| Parameter | Stage | Equation |
| Total | N-8 | (Working Vol. Per Vessel - 10) * (# of Vessels) |
| Volume | N-7 | (Working Vol. Per Vessel) * (1 - Evaporative Loss Fraction) - Sampling Loss |
| After Loss | through | |
| | N-1 | |
| Minimum Final VCD Required | N-8 through N-4 | $If : ( ( \frac{\begin{matrix}Working Vol . Per \\ Vessel of Next Stage\end{matrix}}{Total Vol . After Loss} ) * ( \# Vessels of Next Stage ) ) < 3 ,$ |
| | | then: (Initial VCD of Next Stage) * 3, |
| | | $else : ( \frac{Initial VCD of Next Stage}{Total Vol . After Loss} ) * ( \begin{matrix}Working Vol . Per \\ Vessel of Next Stage\end{matrix} ) * ( \# Vessels of Next Stage )$ |
| | N-3 | $If : ( ( \frac{( \frac{Min . Final VCD Required of Next Stage}{{e}^{( \begin{matrix}Standard Culture \\ Time of Next Stage\end{matrix} ) * ( \begin{matrix}Normal Growth \\ Constant of Next Stage\end{matrix} )}} )}{( Working Vol . Per Vessel ) * ( \# Vessels )} ) * ( \begin{matrix}Working Vol . Per \\ Vessel of Next Stage\end{matrix} ) * ( \frac{\# Vessels of Next Stage}{Initial VCD of Next Stage} ) ) < 3 ,$ |
| | | then: (Initial VCD of Next Stage) * 3, |
| | | $else : ( ( \frac{( \frac{Min . Final VCD Required of Next Stage}{{e}^{( \begin{matrix}Standard Culture \\ Time of Next Stage\end{matrix} ) * ( \begin{matrix}Normal Growth \\ Constant of Next Stage\end{matrix} )}} )}{( Working Vol . Per Vessel ) * ( \# Vessels )} ) * ( Working Vol . Per Vessel ) * ( \# Vessels of Next Stage ) )$ |
| | N-2 | $If : ( ( \begin{matrix}Batch Medium Vol . \\ of Next Stage\end{matrix} ) + ( \begin{matrix}Total Vol . \\ After Loss\end{matrix} ) ) * ( \frac{\# Vessels of Next Stage}{Total Vol . After Loss} ) < 3$ |
| | | then: (Initial VCD of Next Stage) * 3, |
| | | $else : ( ( \begin{matrix}Initial VCD of \\ Next Stage\end{matrix} ) * ( \begin{matrix}Batch Medium Vol . \\ of Next Stage\end{matrix} ) * ( \frac{\# Vessels of Next Stage}{Total Vol . After Loss} )$ |
| | N-1 | $If : ( ( \frac{\begin{matrix}Working Vol . Per \\ Vessel of Next Stage\end{matrix}}{Total Vol . After Loss} ) * ( \# Vessels of Next Stage ) ) < 3 ,$ |
| | | then: (Initial VCD of Next Stage) * 3, |
| | | $else : ( \frac{\begin{matrix}Initial VCD of \\ Next Stage\end{matrix}}{Total Vol . After Loss} ) * ( \begin{matrix}Working Vol . Per \\ Vessel of Next Stage\end{matrix} ) * ( \# Vessels of Next Stage )$ |
| Predicted | N-8 | (Initial VCD) * e^{((Normal Growth Constant)*(Standard Culture Time))} |
| Final VCD | through | |
| | N-1 | |
| Split Ratio | N-8 through N-1 | $\frac{( ( \begin{matrix}Minimum Final \\ VCD Required\end{matrix} ) - ( \begin{matrix}Initial VCD of \\ Next Stage\end{matrix} ) )}{Initial VCD of Next Stage}$ |
| Time Needed - Normal | N-8 through N-1 | $\frac{ln ( \frac{( \begin{matrix}Minimum Final \\ VCD Required\end{matrix} ) * ( ( \begin{matrix}Total Vol . \\ After Loss\end{matrix} ) + 5 )}{( Working Vol . Per Vessel ) * ( \# Vessels ) * ( Initial VCD )} )}{Worst Growth Constant}$ |

TABLE 4
Equations for estimating outcomes of one or more stages of a seed train process.

| Predicted | Equation |
|---|---|
| Outcome | |
| Extra Time | Time Needed_{Normal} - Standard Culture Time |
| Needed - Normal | |
| Extra Time Needed - Worst | $\frac{ln ( \frac{( \begin{matrix}Minimum Final \\ VCD Required\end{matrix} ) * ( ( \begin{matrix}Total Vol . \\ After Loss\end{matrix} ) + 5 )}{( Working Vol . Per Vessel ) * ( \# Vessels ) * ( Initial VCD )} )}{Worst Growth Constant} - Standard Culture Time$ |
| Final VCD - | e^{(Worst Growth Constant*Best Growth Constant)} * Initial VCD |
| Best | |

FIG. 6 shows example historical cell growth data, according to some embodiments of the technology described herein. The historical cell growth data includes data from multiple seed train processes that have already been implemented. The data includes, for each past seed train process, the doubling time of cells grown during a particular stage in the seed train process.

As described herein, in some embodiments, doubling times for a particular stage of a seed train process may be randomly sampled from a distribution of doubling times, such as the one shown in FIG. 6. For example, doubling times may be sampled when determining a likelihood indicative of whether performing the seed train process using determined seed train process parameters will satisfy seed train process success criteria, as described herein including at least with respect to FIG. 2.

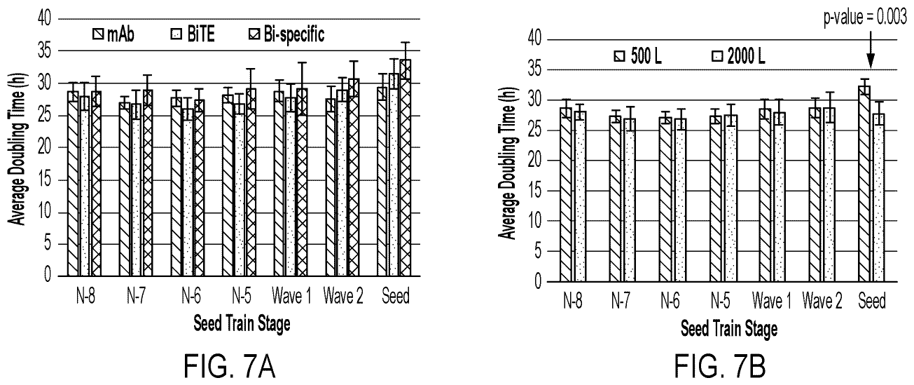

FIG. 7A shows example doubling time data measured from cultures that were grown for producing different molecules including monoclonal antibodies (mAbs), bi-specific T-cell engagers (BiTEs), and bi-specific antibodies. The doubling time data includes an average doubling time and standard deviation for each stage of multiple stages of a seed train process for each culture type (e.g., mAb, BiTE, bi-specific). FIG. 7B shows example doubling time data measured from cultures that were expanded to 500 L containers and cultures that were grown to 2000 L containers. The doubling time data includes an average doubling time and standard deviation for each stage of multiple stages of a seed train process for each container size (e.g., 500 L, 2000 L).

In some embodiments, the doubling time data may be stored and/or used as historical cell growth data. For example, as described herein, the doubling time data may be sampled for each stage and used for predicting the likelihood of satisfying at least one success criterion if the culture were to have the sampled doubling time. Additionally, or alternatively, the doubling time data may be used to specify seed train process constraints. For example, the average doubling time for a particular stage may be specified as the normal doubling time constraint for that stage in the seed train process.

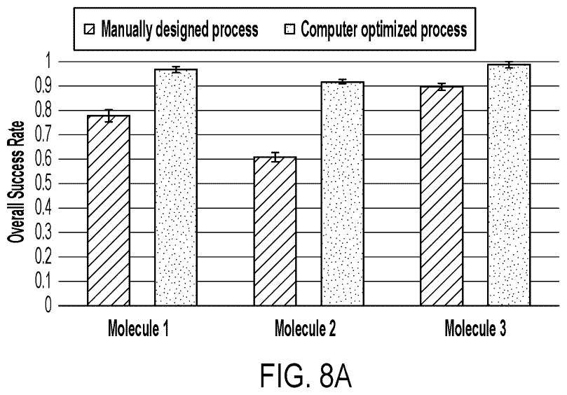

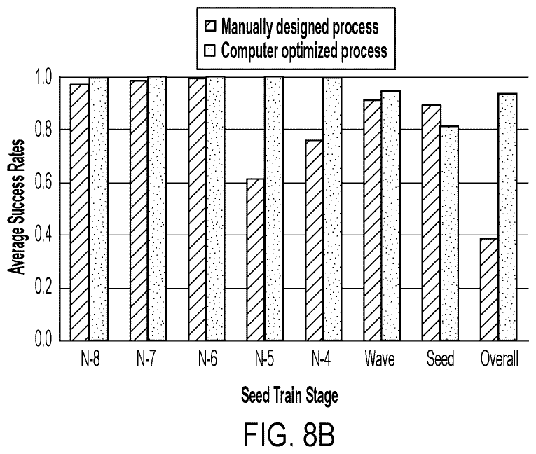

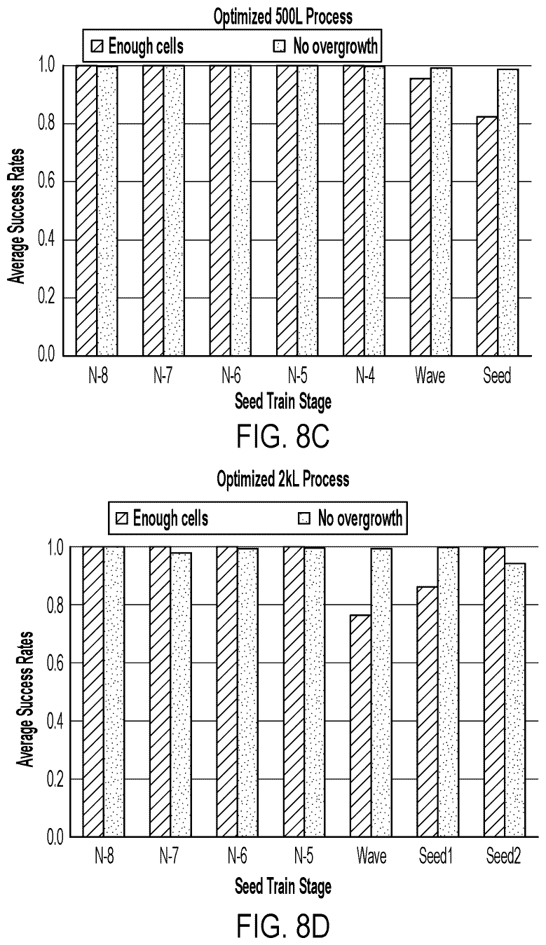

FIG. 8A and FIG. 8B show a higher overall success rate of performing a seed train process, according to embodiments of the technology described herein, as compared to performing a manually-designed seed train process. Accordingly, using the techniques described herein to design a seed train process will help to reduce facility scheduling issues caused by seed train processes that take too long or need to be repeated. Furthermore, it will help to limit waste caused by failed seed train processes that need to be discarded and repeated.

FIG. 8A is a plot comparing the overall success rate of implementing seed train processes that were manually designed to those that were designed according to the techniques described herein. In particular, seed train processes were designed for growing cultures for the production of three different molecules (e.g., molecule 1, molecule 2, and molecule 3). As shown, for each of the molecules, seed train processes that were designed according to the techniques described herein had a higher overall success rate than those that were manually designed.

FIG. 8B is a plot comparing the overall success rate of implementing each stage of seed train processes that were manually designed to those that were designed according to the techniques described herein. As shown, at least seven out of eight of the stages of the seed train processes designed according to the techniques described herein had an equal or higher overall success rate than those that were manually designed.

FIG. 8C shows that performing a 500 L seed train process, according to embodiments of the technology described herein, results in an average success rate of over 80% for each stage of the seed train process. As shown, the successful implementation of a stage means that there were enough cells and there was no overgrowth.

FIG. 8D shows that performing a 2 kL seed train process, according to embodiments of the technology described herein, results in a success rate of over 70% for each stage of the seed train process. As shown, the successful implementation of a stage means that there were enough cells and there was no overgrowth.

An illustrative implementation of a computer system 900 that may be used in connection with any of the embodiments of the technology described herein (e.g., such as the processes of FIGS. 2-3A and 4) is shown in FIG. 9. The computer system 900 includes one or more processors 910 and one or more articles of manufacture that comprise non-transitory computer-readable storage media (e.g., memory 920 and one or more non-volatile storage media 930). The processor 910 may control writing data to and reading data from the memory 920 and the non-volatile storage device 930 in any suitable manner, as the aspects of the technology described herein are not limited to any particular techniques for writing or reading data. To perform any of the functionality described herein, the processor 910 may execute one or more processor-executable instructions stored in one or more non-transitory computer-readable storage media (e.g., the memory 920), which may serve as non-transitory computer-readable storage media storing processor-executable instructions for execution by the processor 910.

Computer device 900 may also include a network input/output (I/O) interface 940 via which the computing device may communicate with other computing devices (e.g., over a network), and may also include one or more user I/O interfaces 950, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-described functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-described functions of one or more embodiments. The computer-readable medium may be transportable such that the program stored thereon can be loaded onto any computing device to implement aspects of the techniques described herein. In addition, it should be appreciated that the reference to a computer program which, when executed, performs any of the above-described functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques described herein.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures. Further, certain portions of the implementations may be implemented as a “module” that performs one or more functions. This module may include hardware, such as a processor, an application-specific integrated circuit (ASIC), or a field-programmable gate array (FPGA), or a combination of hardware and software.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately,” “substantially,” and “about” may be used to mean within ±20% of a target value in some embodiments, within ±10% of a target value in some embodiments, within ±5% of a target value in some embodiments, within ±2% of a target value in some embodiments. The terms “approximately,” “substantially,” and “about” may include the target value.

What is claimed is:

1. A method for determining seed train process parameters for a seed train process for growing a culture of cells, the method performed using at least one software application program comprising a common interface module, a parameter optimization module, and a simulation module, the method comprising:
using at least one computer hardware processor to perform:
obtaining, using the common interface module, a specification of seed train process constraints for multiple stages of the seed train process;
determining the seed train process parameters using the parameter optimization module and the seed train process constraints, the seed train process parameters comprising a respective set of parameters for each particular stage of the multiple stages of the seed train process, the determining comprising:
determining, for each particular stage of the multiple stages and using the parameter optimization module and the seed train process constraints, the respective set of parameters for growing the culture during the particular stage;
determining, using the simulation module, historical cell growth data, and the determined seed train process parameters, a likelihood indicative of whether performing the seed train process using the determined seed train process parameters will satisfy seed train process success criteria; and
outputting the respective set of seed train process parameters determined for each stage of the multiple stages of the seed train process and the likelihood indicative of whether performing the seed train process using the determined seed train process parameters will satisfy the seed train process success criteria.

2. The method of claim 1, wherein determining, for each particular stage of the multiple stages, the respective set of parameters for growing the culture during the particular stage comprises:
determining at least one seed train process parameter selected from the group consisting of: a viable cell density, an indication of whether the particular stage is included in the seed train process, a targeted duration of the seed train process, a batch medium volume, a working volume per vessel, and a number of vessels.

3. The method of claim 1 or any other preceding claim, wherein obtaining the specification of the seed train process constraints comprises:
obtaining a specification of at least one seed train process constraint selected from the group consisting of: a target viable cell density range, a target working volume range, a split ratio culture growth process constraint, and a batch media volume range.

4. The method of claim 1 or any other preceding claim, wherein the historical cell growth data comprises data indicative of a doubling time associated with the growth of one or more cultures of cells.

5. The method of claim 1 or any other preceding claim, wherein determining the seed train process parameters using the parameter optimization module comprises determining the seed train process parameters using a genetic algorithm.

6. The method of claim 1 or any other preceding claim, wherein determining the likelihood indicative of whether performing the seed train process using the determined seed train process parameters will satisfy the seed train process success criteria comprises performing Monte Carlo simulations using the historical cell growth data.

7. The method of claim 1 or any other preceding claim, wherein the seed train process success criteria comprise, for each stage of the multiple stages of the seed train process:
a first criterion that an expected duration needed to grow the culture during the particular stage does not exceed a threshold duration for growing the culture during the particular stage; and
a second criterion that an expected viable cell density resulting from growing the culture during the particular stage does not exceed a threshold viable cell density resulting from growing the culture during the particular stage.

8. The method of claim 7, wherein determining the respective set of parameters for each particular stage of the multiple stages comprises:
determining, using the respective set of parameters, the expected duration needed to grow the culture of cells during the particular stage; and
determining whether the expected duration exceeds the respective threshold duration for the particular stage.

9. The method of claim 7, wherein determining the respective set of parameters for each particular stage of the multiple stages comprises:
determining, using the respective set of parameters, the expected viable cell density resulting from growing the culture of cells during the particular stage; and
determining whether the expected viable cell density exceeds the respective threshold viable cell density for the particular stage.

10. The method of claim 1 or any other preceding claim, wherein outputting the set of seed train process parameters for each stage of the multiple stages of the seed train process and the likelihood indicative of whether performing the seed train process will satisfy the seed train process success criteria comprises:
generating a graphical user interface (GUI) using the common interface module; and
displaying the set of seed train process parameters and/or the likelihood through the generated GUI.

11. The method of claim 10, wherein displaying the set of seed train process parameters through the common interface module comprises:
displaying a visual indication of whether a seed train process parameter of the set of seed train process parameters violates a seed train process constraint of the seed train process constraints.

12. The method of claim 1 or any other preceding claim,
wherein determining the seed train process parameters comprises:
determining the seed train process parameters based on a set of estimate culture doubling times included in the seed train process constraints, the set of estimate culture doubling times including an estimate culture doubling time for each of the multiple stages of the seed train process; and
determining whether performing the seed train process using the determined seed train process parameters will satisfy the seed train process success criteria, and wherein the method further comprises:
displaying, through a graphical user interface (GUI) generated by the common interface module, a visual indication of a result of determining whether performing the seed train process using the seed train process parameters will satisfy the seed train process success criteria.

13. The method of claim 1 or any other preceding claim, further comprising:
comparing the determined likelihood to a likelihood threshold, wherein outputting the set of seed train process parameters for each stage of the multiple stages of the seed train process comprises outputting determined the seed train process parameters upon determining that the likelihood exceeds the likelihood threshold.

14. The method of claim 1 or any other preceding claim, wherein outputting the set of seed train process parameters for each stage of the multiple stages of the seed train process comprises:
when the likelihood indicative of whether performing the seed train process using the determined seed train process parameters will satisfy the seed train process success criteria satisfies a likelihood threshold, outputting a recommendation to perform the seed train process using the set of seed train process parameters for each stage of the multiple stages of the seed train process.

15. The method of claim 1 or any other preceding claim, wherein the at least one software application program further comprises a seed train process automation module, and wherein outputting the set of seed train process parameters for each stage of the multiple stages of the seed train process and the likelihood indicative of whether performing the seed train process will satisfy the seed train process success criteria comprises:
transmitting the set of seed train process parameters and/or the likelihood to the seed train process automation module; and
using the seed train process automation module to cause a seed train process automation system to perform a particular stage of the multiple stages of the seed train process according to the respective set of seed train process parameters.

16. The method of claim 1 or any other preceding claim, further comprising:
obtaining, using the common interface module, input indicative of a result of growing the culture of cells during a first stage of the multiple stages of the seed train process;
determining, using the parameter optimization module, the input, and the seed train process constraints, updated seed train process parameters for subsequent stages of the seed train process; and
outputting the updated seed train process parameters.

17. The method of claim 1 or any other preceding claim, further comprising:
obtaining, using the common interface module, a specification of second seed train process constraints for multiple stages of a second seed train process;
determining, using the parameter optimization module and the second seed train process constraints, second seed train process parameters for the second seed train process;
determining, using the simulation module, the historical cell growth data, and the second seed train process parameters, a second likelihood indicative of whether performing the second seed train process, using the second seed train process parameters, will satisfy second seed train process criteria; and
outputting the second seed train process parameters and the second likelihood indicative of whether performing the second seed train process, using the second seed train process parameters, will satisfy the second seed train process criteria.

18. The method of claim 1 or any other preceding claim, wherein determining the seed train process parameters using the parameter optimization module comprises:
determining, using an objective function, a first score for first candidate seed train process parameters;
determining, using the objective function, a second score for second candidate seed train process parameters;
comparing the first score and the second score; and
selecting, based on a result of the comparing, the seed train process parameters from among the first candidate seed train process parameters and the second candidate seed train process parameters.

19. The method of claim 1 or any other preceding claim,
wherein the seed train process parameters comprise a first set of seed train process parameters for a first stage of the multiple stages of the seed train process, and
wherein determining the likelihood indicative of whether performing the seed train process using the seed train process parameters will satisfy the seed train process success criteria comprises:
determining, for the first stage of the seed train process, a first likelihood indicative of whether growing the culture of cells during the first stage, using the first set of seed train parameters for the first stage, will satisfy first criteria of the seed train process success criteria.

20. The method of claim 1 or any other preceding claim, wherein determining the likelihood indicative of whether performing the seed train process using the seed train process parameters will satisfy the seed train process success criteria comprises:
simulating a first set of cell growth values using the historical cell growth data, the first set of cell growth values including a first historical cell growth value for each of the multiple stages of the seed train process;
predicting, using the seed train process parameters and the first set of cell growth values, a first outcome of performing the seed train process, wherein the first outcome is indicative of whether performing the seed train process using the determined seed train process parameters will satisfy the seed train process success criteria;
simulating a second set of cell growth values using the historical cell growth data, the second set of cell growth values including a second historical cell growth value for each of the multiple stages of the seed train process;
predicting, using the seed train process parameters and the second set of cell growth values, a second outcome of performing the seed train process, wherein the second outcome is indicative of whether performing the seed train process using the seed train process parameters will satisfy the seed train process success criteria; and
determining the likelihood based on the predicted first and second outcomes.

21. The method of claim 20, wherein determining the likelihood based on the predicted first and second outcomes comprises:
determining a number of predicted outcomes that indicate that performing the seed train process using the seed train process parameters and respective historical cell growth data will satisfy the seed train process success criteria; and
determining a ratio of the number of predicted outcomes to a total number of predicted outcomes.

22. The method of claim 20,
wherein the seed train process success criteria comprise, for each stage of the multiple stages of the seed train process:
a first criterion that an expected duration needed to grow the culture during the particular stage does not exceed a threshold duration for growing the culture during the particular stage,
wherein predicting the first outcome comprises, for each particular stage of the multiple stages:
determining, using the respective set of seed train process parameters for the particular stage and a historical cell growth value of the first set of historical cell growth values, a first expected duration needed to grow the culture of cells during the particular stage of the seed train process; and
comparing the first expected duration to the threshold duration, and
wherein predicting the second outcome comprises, for each particular stage of the multiple stages:
determining, using the respective set of seed train process parameters for the particular stage and a historical cell growth value of the second set of historical cell growth values, a second expected duration needed to grow the culture of cells during the particular stage of the seed train process; and
comparing the second expected duration to the threshold duration.

23. The method of claim 20,
wherein the seed train process success criteria comprise, for each stage of the multiple stages of the seed train process:
a second criterion that an expected viable cell density resulting from growing the culture during the particular stage does not exceed a threshold viable cell density resulting from growing the culture during the particular stage,
wherein predicting the second outcome comprises, for each particular stage of the multiple stages:
determining, using the respective set of seed train process parameters for the particular stage and a historical cell growth value of the first set of historical cell growth values, a first expected viable cell density resulting from growing the culture of cells during the particular stage of the seed train process; and
comparing the first expected viable cell density to the threshold viable cell density, and
wherein predicting the second outcome comprises, for each particular stage of the multiple stages:
determining, using the respective set of seed train process parameters for the particular stage and a historical cell growth value of the second set of historical cell growth values, a second expected viable cell density resulting from growing the culture of cells during the particular stage of the seed train process; and
comparing the second expected viable cell density to the threshold viable cell density.

24. A system, comprising:
at least one computer hardware processor; and
at least one non-transitory computer-readable storage medium storing processor-executable instructions that, when executed by the at least one computer hardware processor, cause the at least one computer hardware processor to perform the method of any one of claims 1-23.

25. At least one non-transitory computer-readable storage medium storing processor-executable instructions that, when executed by at least one computer hardware processor, cause the at least one computer hardware processor to perform the method of any one of claims 1-23.
